SEQUENCE LISTING (14 sequences)

SEQ ID NO: 1 - DNA, 492 bp, Homo sapiens
  source 1..492: mol_type = other DNA
atggggatccttcccagccctgggatgcctgcgctgctctccctcgtgagccttctctccgtgctgctgatgggttgcgtagctgaaaccggtgcccccaccagctccacaaagaagacccagctgcagctggagcacctgctgctggacctgcagatgatcctgaacggcatcaacaattacaagaatccaaagctgacacggatgctgaccttcaagttttatatgcccaagaaggccacagagctgaagcacctgcagtgcctggaggaggagctgaagcctctggaggaggtgctgaacctggcccagtccaagaatttccacctgcggccaagagacctgatctctaacatcaatgtgatcgtgctggagctgaagggcagcgagaccaccttcatgtgcgagtacgccgatgagaccgccacaatcgtggagttcctgaacaggtggatcaccttttgtcagtccatcatctctaccctgacatga

SEQ ID NO: 2 - protein, 163 aa, Homo sapiens
MGILPSPGMPALLSLVSLLSVLLMGCVAETGAPTSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 3 - protein, 263 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 4 - protein, 45 aa, Homo sapiens
TTTPAPRPPTPAPTIASQPLSLRPEACRPAAGGAVHTRGLDFACD

SEQ ID NO: 5 - protein, 24 aa, Homo sapiens
IYIWAPLAGTCGVLLLSLVITLYC

SEQ ID NO: 6 - protein, 62 aa, Homo sapiens
KKVAKKPTNKAPHPKQEPQEINFPDDLPGSNTAAPVQETLHGCQPVTQEDGKESRISVQERQ

SEQ ID NO: 7 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 243 aa, Homo sapiens
KGLAPAEISAVCEKGNFNVAHGLAWSYYIGYLRLILPELQARIRTYNQHYNNLLRGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 9 - DNA, 8699 bp, synthetic construct
  source 1..8699: mol_type = other DNA
caatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgacgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggttaacttttaaaagaaaaggggggattggggggtacagtgcaggggaaagaatagtagacataatagcaacagacatacaaactaaagaattacaaaaacaaattacaaaaattcaaaattttatcgatactagttgaattagtttcactttccagtttcagtttccagtttcattttccagtttcattttccagtttcattttcctgatatcctgcaggagcttgaataaaatgaatattagaagctgttagaataagagaaaatgacagaggaaaactgaaagggagaactgaaagtgggaaattcctctgaggcagaaaggaccatcccttataaatagcacaggccatgaaggaagatcattctcactgcagcctttgacagcctttgcctcatcttgggatccaccatggggatccttcccagccctgggatgcctgcgctgctctccctcgtgagccttctctccgtgctgctgatgggttgcgtagctgaaaccggtgcccccaccagctccacaaagaagacccagctgcagctggagcacctgctgctggacctgcagatgatcctgaacggcatcaacaattacaagaatccaaagctgacacggatgctgaccttcaagttttatatgcccaagaaggccacagagctgaagcacctgcagtgcctggaggaggagctgaagcctctggaggaggtgctgaacctggcccagtccaagaatttccacctgcggccaagagacctgatctctaacatcaatgtgatcgtgctggagctgaagggcagcgagaccaccttcatgtgcgagtacgccgatgagaccgccacaatcgtggagttcctgaacaggtggatcaccttttgtcagtccatcatctctaccctgacatgaactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagattctagagctagcgatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccgtgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttgagtcga

SEQ ID NO: 10 - DNA, 732 bp, Homo sapiens
  source 1..732: mol_type = genomic DNA
aagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccgtgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 11 - protein, 258 aa, synthetic construct
KGLAPAEISAVCEKGNFNVAHGLAWSYYIGYLRLILPELQARIRTYNQHYNNLLRGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDPVETTDYFNPSSAMKQN

SEQ ID NO: 12 - DNA, 777 bp, synthetic construct
  source 1..777: mol_type = other DNA
aagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccgtgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatcctgtggagaccaccgattattttaacccatctagcgcaatgaaacaaaactaa

SEQ ID NO: 13 - DNA, 267 bp, Homo sapiens
  source 1..267: mol_type = genomic DNA
tgaattagtttcactttccagtttcagtttccagtttcattttccagtttcattttccagtttcattttcctgatatcctgcaggagcttgaataaaatgaatattagaagctgttagaataagagaaaatgacagaggaaaactgaaagggagaactgaaagtgggaaattcctctgaggcagaaaggaccatcccttataaatagcacaggccatgaaggaagatcattctcactgcagcctttgacagcctttgcctcatcttg

SEQ ID NO: 14 - DNA, 157 bp, Homo sapiens
  source 1..157: mol_type = genomic DNA
ggggactttccgggaatttccggggactttccgggaatttccgggaatttccggggactttccgggaatttccggggactttccgggaatttccagatctggcctcggcggccaagcttgctagcggggggctataaaagggggtgggggcgttcgt

(19) United States
(12) Patent Application Publication
(10) Pub. No.: US 2026/0347773 A1
(43) Pub. Date: Nov. 5, 2026

(54) Myeloid cells expressing IL-2 and uses thereof for quick anticancer therapy

(71) Applicants: INSTITUT CURIE, Paris (FR); INSTITUT NATIONAL DE LA SANTE ET DE LA RECHERCHE MEDICALE, Paris (FR)

(72) Inventors: Philippe BENAROCH, Paris (FR); Jovan NIKOLIC, Montrouge (FR); Nadia JEREMIAH, Vanves (FR); Eliane PIAGGIO, Paris (FR); Christine SEDLIK, Le Plessis Bouchard (FR)

(21) Appl. No.: 19/114,568

(22) PCT Filed: Sep. 26, 2023
(86) PCT No.: PCT/EP2023/076524

§ 371 Date: Mar. 24, 2025

(30) Foreign Application Priority Data
Sep. 26, 2022 (EP) 22306417.1

(51) Int. Cl. C12N 5/0783; A61K 40/11; A61K 40/31; A61K 40/35; A61K 40/42; A61P 35/00; C07K 14/55; C07K 16/28

(52) U.S. Cl. CPC C12N 5/0636; A61K 40/11; A61K 40/31; A61K 40/35; A61K 40/4211; A61K 40/4234; A61P 35/00; C07K 14/55; C07K 16/2803; C12N 2510/00

(57) ABSTRACT

The present invention concerns a modified cell comprising a transgene coding for interleukin-2 (IL-2). Said modified cell comprises a first vector comprising a sequence coding for IL-2 under the control of an inducible or constitutive promoter, which notably enables a locally contained expression of IL-2. It also relates to therapeutic uses thereof.

The present invention concerns a modified cell comprising a transgene coding for interleukin-2 (IL-2). Said modified cell, preferably modified myeloid cell, comprises a first vector comprising a sequence coding for IL-2 under the control of an inducible or constitutive promoter, which notably enables a locally contained expression of IL-2. It also relates to therapeutic uses thereof.

Solid tumors and their metastases are the most common and therapeutically challenging types of cancer today. The tumor microenvironment (TME) is a complex, heterogeneous mix of cellular populations that interact with one another and with the tumor cells. The TME is immunosuppressive, both evading the immune system and preventing therapeutic intervention from efficiently eliminating malignant cells. Myeloid cells within the TME play an important role in contributing to immune evasion by exhibiting potent immunosuppressive as well as pro-tumorigenic properties.

TAMs (tumor-associated macrophages) are a key cell component of the TME in a variety of cancers. The prevailing consensus is that tumor-derived cytokines direct myeloid cell recruitment at the monocyte stage, and then the TME influences their development into polarized macrophages. TAMs can represent a significant portion of the tumor mass, up to 50% in some breast tumors. They develop into immunosuppressive macrophages, which hinder anti-tumor CD8+ T cells from infiltrating the tumor and attract or induce regulatory T cells (Treg). TAMs secrete growth factors like VEGF or TGFβ, which promote tumor growth and invasive behavior. They are generally associated with poor prognosis, though recent studies have shown that their impact on prognosis can vary depending on their localization and polarization (Ramos et al, 2022, Cell 185, 1-19, Tissue-resident FOLR2+ macrophages associate with tumor-infiltrating CD8+ T cells and with increased survival of breast cancer patients).

Impressive successes have been recently obtained to treat certain malignancies with autologous immune cell-based therapies. The most advanced approaches rely on T lymphocytes that have been genetically modified to express chimeric receptors that combine antigen-binding and T-cell activation activities in a single receptor are known as CAR (Chimeric Antigen Receptor) T cells. Adoptively transplanted CAR-T cells have shown considerable promise in fighting hematological malignancies.

CAR-T cell treatments, however, have so far failed to treat solid tumors. These failures probably result from a combination of factors. First, identifying antigens strictly tumor-specific remains difficult, raising concerns about potential off-target effects. Second, before reaching the cancer cells within the tumor tissue, the CAR-T cells may encounter physical barriers in the form of TAMs and cancer-associated fibroblasts that produce vast amounts of extracellular matrix. Third, CAR-T cells do not successfully invade the TME due to a lack of metabolic resources or signals provided by TME cell components. Finally, due to persistent antigen stimulation they receive via tumor cells, CAR-T cells become “exhausted” or dysfunctional losing their effector function and failing to evolve into effector memory T cells.

Macrophages are antigen-presenting cells that can stimulate T cells locally and thus promote adaptive anti-tumor responses. Macrophages produce proteases that can dramatically modify the extracellular matrix within the tumor mass and hence the architecture of the tumor tissue. Macrophages also have anti-tumor capabilities, such as the ability to phagocyte entire tumor cells or undertake antibody-dependent cell phagocytosis. The intrinsic features of macrophages make them an ideal candidate to overcome the limitations of CAR-T cells.

However, as macrophages also tend to reach tumor tissue with difficulties, there is thus a need for improving the access of immune cells into the tumor tissues. In this regard, monocytes are considered as a better candidate for cell modification due to their better ability to penetrate the tumor tissue before differentiation into macrophages.

Systemic administration of IL-2 to boost the T cell responses promote anti-tumor immunity in mouse tumor models. The cytokine IL-2 has pleiotropic effects activating T and NK cells. However, systemic administration of IL-2 needs repetitive injection as IL-2 has a short half-life in vivo. These repeated injections have been shown to have deleterious effects. IL-2 has therefore been neglected in therapeutic uses, but local expression of IL-2 could be of major interest.

Thus, there is a need for a specific expression of IL-2 into tumor (in situ), which could be helpful to recruit local immune cells

The present invention fulfills these needs.

### SUMMARY OF THE INVENTION

The present invention thus relates to modified cell, wherein the cell is chosen from myeloid cells, induced pluripotent stem cells (iPS) and hematopoietic stem cells (HSC), and wherein the cell comprises:
(i) either a first vector comprising a sequence coding for at least one cytokine, preferably at least one interleukin, under the control of an inducible or constitutive promoter, and a second vector comprising a sequence coding for a chimeric antigen receptor (CAR); or
(ii) only comprises the first vector, which encodes for both the at least one cytokine, preferably at least one interleukin, and for the CAR.

The present invention thus relates to a modified cell expressing interleukin-2 (IL-2), wherein the cell is chosen from myeloid cells, induced pluripotent stem cells (iPS) and hematopoietic stem cells (HSC), and wherein the cell comprises a first vector comprising a sequence coding for IL-2 under the control of an inducible or constitutive promoter. Said cell is called the modified myeloid cell, the modified iPS or the modified HSC according to the invention.

Preferably, the modified cell is a modified myeloid cell which comprises a first vector comprising a sequence coding for IL-2 under the control of an inducible or constitutive promoter.

Preferably, the modified cell further comprises a second vector comprising a sequence coding for a chimeric antigen receptor (CAR).

The invention also relates to a modified cell expressing at least one cytokine, preferably an interleukin, wherein the cell is chosen from myeloid cells, induced pluripotent stem cells (iPS) and hematopoietic stem cells (HSC), and wherein the cell comprises a first vector comprising a sequence coding for said cytokine under the control of an inducible or constitutive promoter, and optionally a second vector comprising a sequence coding for a chimeric antigen receptor (CAR),
wherein said CAR comprises:
   an extracellular antigen-binding domain which binds to a tumor antigen or an antigen present on cells of the tumor microenvironment (TME);
   optionally a hinge domain;
   a transmembrane domain, and
   an intracellular signaling domain.

The invention also relates to a modified cell expressing at least one interleukin chosen from IL-10, IL-15, IL-13, IL-7A, IFNalpha, IFNbeta, IFNlambda, IFNgamma, IL-1A, IL-1B, IL-12 and IL-21, wherein the cell is chosen from myeloid cells, induced pluripotent stem cells (iPS) and hematopoietic stem cells (HSC), and wherein the cell comprises a first vector comprising a sequence coding for said interleukin under the control of an inducible or constitutive promoter, and optionally a second vector comprising a sequence coding for a chimeric antigen receptor (CAR),
wherein said CAR comprises:
   an extracellular antigen-binding domain which binds to a tumor antigen or an antigen present on cells of the tumor microenvironment (TME);
   optionally a hinge domain;
   a transmembrane domain, and
   an intracellular signaling domain.

In some embodiments, the at least one cytokine, preferably an interleukin, is linked with a 2A peptide to another cytokine, preferably another interleukin, an antigenic polypeptide, a single chain antibody or a nanobody, which allows for ribosomal skipping during translation of a protein. Said antigenic polypeptide, single chain antibody or nanobody can either block or stimulate a receptor and allow to target specific cells, preferably tumor cells or cells from the TME such as cancer-associated fibroblast (CAF), or regulatory T cells. Preferably, said antigenic polypeptide has a length of about 8 to about 50 amino acids.

The present invention also relates to a method for manufacturing a modified myeloid cell, a modified iPS or a modified HSC, the method comprising:
providing at least one cell chosen from isolated myeloid cells, iPS and HSC;
transducing said cell with a first vector, preferably a lentiviral vector, comprising a cytokine, preferably an interleukin, under the control of an inducible or constitutive promoter, preferably under the control of the promoter of interleukin-6 or the promoter of interleukin-8; and
optionally, transducing said cell with a second vector, preferably a lentiviral vector, comprising a nucleic sequence coding for a CAR.

The present invention also relates to a method for manufacturing a modified myeloid cell, a modified iPS or a modified HSC, the method comprising:
providing at least one cell chosen from isolated myeloid cells, iPS and HSC;
transducing said cell with a first vector, preferably a lentiviral vector, comprising a sequence coding for IL-2 or a sequence coding for at least one interleukin chosen from IL-10, IL-15, IL-13, IL-7A, IFNalpha, IFNbeta, IFNlambda, IFNgamma, IL-1A, IL-1B, IL-12 and IL-21, under the control of an inducible or constitutive promoter, preferably under the control of the promoter of interleukin-6 or the promoter of interleukin-8; and
optionally, transducing said cell with a second vector, preferably a lentiviral vector, comprising a nucleic sequence coding for a CAR.

The present invention also relates to a pharmaceutical composition comprising the modified myeloid cell, the modified iPS or the modified HSC according to the invention, and a pharmaceutical acceptable carrier.

The present invention also relates to the use of the modified myeloid cell, the modified iPS or the modified HSC according to the invention, or of the pharmaceutical composition, in the treatment of cancer, an autoimmune disease or an inflammatory disease.

The present invention further relates to products containing the modified myeloid cell, the modified iPS or the modified HSC according to the invention, and a CAR-T cell, as a combined preparation for simultaneous, separate or sequential use in treatment of cancer, an autoimmune disease or an inflammatory disease.

The present invention also relates to products containing the modified myeloid cell, the modified iPS or the modified HSC according to the invention, and an Immune Checkpoint Inhibitor as a combined preparation for simultaneous, separate or sequential use in treatment of cancer, an autoimmune disease or an inflammatory disease.

### DETAILED DESCRIPTION OF THE INVENTION

Surprisingly, as shown in the examples, the inventors have demonstrated that monocytes which are transduced both with a specific CAR and with the gene coding for IL-2 under the control of a specific promoter, may be used as treatment notably in cancer therapy.

Indeed, said monocytes are able to penetrate tumor and differentiate as IL-2 expressing macrophages able to bind a given antigen, activating thus their great capacity to phagocytose tumor cells, but also to further mobilize the antigen presentation and co-stimulatory capacities of macrophages upon their encounter of tumor cells, and thus to stimulate anti-tumor immunity.

The present invention relates to modified cell, wherein the cell is chosen from myeloid cells, induced pluripotent stem cells (iPS) and hematopoietic stem cells (HSC), and wherein the cell comprises:
(i) either a first vector comprising a sequence coding for at least one cytokine, preferably at least one interleukin, under the control of an inducible or constitutive promoter, and a second vector comprising a sequence coding for a chimeric antigen receptor (CAR); or
(ii) only comprises the first vector, which encodes for both the at least one cytokine, preferably at least one interleukin, and for the CAR.

### Modified Cell

Preferably the present invention relates to a modified myeloid cell expressing IL-2, wherein the cell comprises a first vector comprising a sequence coding for IL-2 under the control of an inducible or constitutive promoter.

Preferably the present invention also relates to a modified induced pluripotent stem cell (iPS) or hematopoietic stem cell (HSC) expressing interleukin-2 (IL-2), wherein the cell comprises a first vector comprising a sequence coding for IL-2 under the control of an inducible or constitutive promoter.

Said cell is also called the “modified cell” in the present application.

By “myeloid cell”, it is meant any type of cells derived from the myeloid tissue (bone marrow), or resembling bone marrow. Preferably, it is a monocyte, a macrophage or a dendritic cell, more preferably a monocyte. Said myeloid cell is modified in that it expresses at least IL-2.

By “stem cell”, it is meant a cell that, by successive divisions can give rise to specialized cells. The term “pluripotent stem cell” refers to a stem cell that has the potential to differentiate into any of the three germ layers: endoderm (interior stomach lining, gastrointestinal tract, the lungs), mesoderm (muscle, bone, blood, urogenital), or ectoderm (epidermal tissues and nervous system). Pluripotent stem cells can give rise to any fetal or adult cell type but they cannot give rise to an entire organism. A “pluripotent stem cell” may be identified by the expression of one or more of the cell markers Klf4, Sox2, Oct4, cMyc, Nanog and SSEA1. A cell is considered as a pluripotent stem cell when it is capable of generating cells from any of the three germ layers: endoderm, identified by the expression of alpha-fetoprotein; mesoderm (identified by the expression of desmin and/or alpha smooth muscle actin) and ectoderm (identified by the expression of beta-tubulin III=Tuj1 and/or E to N-cadherin). Assays to assess the pluripotentiality of a cell are known in the art.

By “induced pluripotent stem cell” or “iPS”, it is meant a pluripotent cell artificially derived from a non-pluripotent cell, typically an adult somatic cell, by inducing a forced expression of certain genes. An “induced pluripotent stem cell” is defined by the expression of several transcription factors including one or more of Klf4, Sox2, Oct4 and cMyc. iPS cells are typically derived by transfection of certain stem cell-associated genes into non-pluripotent cells, such as adult fibroblasts. Transfection is typically achieved through viral vectors, such as retroviruses, and transfected genes include Oct-3/4 (Pou5fl) and Sox2. Additional genes include certain members of the Klf family (Klfl, Klf2, Klf4 and Klf5), the Myc family (c-myc, L-myc, N-myc), Nanog and LIN28 have been identified to increase the induction efficiency. After 3-4 weeks, small numbers of transfected cells begin to become morphologically and biochemically similar to pluripotent stem cells, and are typically isolated through morphological selection, doubling time, or through a reporter gene and antibiotic selection. Protocols for iPS culture are disclosed in Mochiduki and Okita, 2012. Non-pluripotent cells that can be used to obtain iPS are, without limitation, fibroblasts, keratinocytes and adipocytes. These cells can be obtained from an adult being by methods well-known in the state of the art (Mochiduki and Okita, 2012).

The «hematopoietic stem cells» (HSC) possess the ability to fully reconstitute the immune system of a lethally irradiated host from which the cells are obtained. The hematopoietic stem cells give rise to all blood and immune cells.

Preferably, the cell, especially the myeloid cell of the HSC, is obtained from a biological sample from a donor affected by cancer, an autoimmune disease or an inflammatory disease.

First Vector Coding for at Least One Cytokine, Preferably at Least One Interleukin, Preferably with IL-2

Interleukin-2 (IL-2) is the interleukin that induces the proliferation of responsive T cells and NK cells. IL-2 enhances activation-induced cell death (AICD). IL-2 also promotes the differentiation of T cells into effector T cells and into memory T cells when the initial T cell is also stimulated by an antigen, thus helping the body fight off infections. Together with other polarizing cytokines, IL-2 stimulates naive CD4+ T cell differentiation into Th1 and Th2 lymphocytes while it impedes differentiation into Th17 and folicular Th lymphocytes. IL-2 increases also the cell killing activity of both natural killer cells and cytotoxic T cells.

Preferably, IL-2 is human IL-2 (hlL-2).

Preferably, IL-2 is encoded by the following nucleic sequence:

(SEQ ID NO: 1)
Atggggatccttcccagccctgggatgcctgcgctgctctccctcgtgag
ccttctctccgtgctgctgatgggttgcgtagctgaaaccggtgccccca
ccagctccacaaagaagacccagctgcagctggagcacctgctgctggac
ctgcagatgatcctgaacggcatcaacaattacaagaatccaaagctgac
acggatgctgaccttcaagttttatatgcccaagaaggccacagagctga
agcacctgcagtgcctggaggaggagctgaagcctctggaggaggtgctg
aacctggcccagtccaagaatttccacctgcggccaagagacctgatctc
taacatcaatgtgatcgtgctggagctgaagggcagcgagaccaccttca
tgtgcgagtacgccgatgagaccgccacaatcgtggagttcctgaacagg
tggatcaccttttgtcagtccatcatctctaccctgacatga.

Preferably, IL-2 is encoded by the following amino acid sequence:

(SEQ ID NO: 2)
MGILPSPGMPALLSLVSLLSVLLMGCVAETGAPTSSTKKTQLQLEHLLLD
LQMILNGINNYKNPKLTRMLTFKFYMPKKATELKHLQCLEEELKPLEEVL
NLAQSKNFHLRPRDLISNINVIVLELKGSETTFMCEYADETATIVEFLNR
WITFCQSIISTLT.

The first vector of the modified myeloid cell, the modified iPS or the modified HSC according to the invention comprises a sequence coding for IL-2 under the control of an inducible or constitutive promoter. Preferably, the sequence coding for IL-2 is inserted in a lentivector, preferably pCDH1, optionally containing a resistance gene, and under control of an inducible or constitutive promoter. Preferably, the promoter is inducible.

Constitutive or inducible promoters are usually associated with constitutive or inducible genes, respectively. A constitutive gene is a gene that is permanently transcribed, as opposed to a facultative gene. An inducible gene is a gene whose expression responds to a stimulus, such as an environmental change or the position in the cell cycle. An inducible gene is inactive unless there is the presence of an inducer that allows the gene to be expressed. The use of such constitutive or inducible promoters in genetic engineering allows the regulation of the transduced genes of interest to be controlled. Inducible promoters are generally preferred over constitutive promoters for their reversibility and flexibility. In addition, compared to constitutive promoters, inducible promoters are generally more efficient and have fewer side effects, such as cell death and delayed growth or development.

Preferably, the constitutive promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, such as the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, the actin promoter, the myosin promoter, the hemoglobin promoter and the creatine kinase promoter.

Preferably, the first vector comprises an inducible promoter. Preferably said inducible promoter is specific, i.e. it activates transcription mainly in response to CAR activation. An inducible promoter provides a molecular switch capable of turning on expression of the polynucleotide sequence which it is operatively linked when such expression is desired, or turning off the expression when expression is not desired. Inducible promoters are well-known in the art.

Preferably, the inducible promoter is chosen from positive and negative inducible promoters. In the case of a positive inducible promoter, the activator protein binds to the promoter to initiate transcription. In contrast, in the case of a negative inducible promoter, the promoter is inactive, because a bound repressor protein actively prevents transcription. Once an inducer binds to the repressor protein, said repressor protein is removed from the DNA, and transcription can be activated.

The inducible promoter can be activated in response to a stimulus, such as the presence or absence and/or the amount of a chemical agent, or a change of temperature and/or light.

Chemically regulated promoters are among the most common inducible promoters. The inducible positive tetracycline ON (Tet-On) system, for example, works by direct activation. In this system, the tetracycline-controlled reverse transactivator (rtTA) is normally inactive and cannot bind to tetracycline response elements (TREs) in a promoter. Tetracycline and its derivatives are used as inducing agents to enable promoter activation. The inducible promoter may also be a promoter inducible by a chemical agent whose administration would be controlled over time or even locally. The administered chemical agent can be chosen from a wide possibility of molecules, such as tetracycline, biotin or the combination Tet-Off/Tet-On. The possibility of using photoimmunotherapy would also allow a localized and limited time action.

Cytokine specific promoters are also known; they are activated once the cytokine binds to the promoter. Preferably, the cytokine specific promoter is the promoter of interleukin-6 or the promoter of interleukin-8.

Other examples include the negative inducible promoter pLac or the negative inducible promoter pBad.

Some promoters are temperature inducible. They show almost no expression at normal temperatures but can be induced by exposure to heat or cold. For example, the Hsp70 promoter is inducible by heat shock.

Light is another means of activating gene expression, and two-component systems used in synthetic biology use light to regulate transcription. This requires a light-sensitive protein, such as YF1 (known as Histidine Kinase), that is capable of inducing a transcriptional response, including acting on the synthesis of a repressor or an inducer.

Thus, preferably, the inducible promoter is chosen from chemically regulated promoters, temperature inducible promoters and light inducible promoters. Preferably, the inducible promoter is chosen from cytokine specific promoters (more preferably from the promoter of interleukin-6 and the promoter of interleukin-8), metallothionine promoters, glucocorticoid promoters, progesterone promoters, tetracycline promoters (such as Tet-On promoter), pLac, pBad, heat shock protein promoters (more preferably Hsp70 promoter) and YF1. The inducible promoter may also be chosen from artificial promoters containing response elements that are activated via the activation of the CAR. Artificial promoters may be designed from minimal promoters complemented with multiple binding sites for transcription factors such as NF-KB, AP1 or ISRE (IFN-sensitive response element). Preferably, the artificial promoter comprises at least ISRE. Preferably, the artificial promoter is a mouse INFbeta promoter (that comprises ISRE), a promoter complemented with NF-KB responsive elements, or a combination thereof. The promoter may also be promX, of SEQ ID NO: 13. A preferred inducible promoter is a cytokine specific promoter (more preferably the promoter of interleukin-6 or the promoter of interleukin-8 or the promoter of interferon beta) which comprises response elements (i.e. binding sequences) to NF-KB or ISRE, or both. Preferably, the inducible promoter is promX of SEQ ID NO: 12 or a cytokine-specific promoter which comprises response elements to NF-KB or ISRE, preferably the inducible promoter is promX of SEQ ID NO: 13 or the promoter NF-KB of SEQ ID NO:14.

Said inducible promoter is preferably activated by the tumor environment around the IL-2 expressing cell. Preferably, the inducible promoter is a cytokine specific promoter. Preferably, the cytokine specific promoter is the promoter of interleukin-6 or the promoter of interleukin-8 or a synthetic promoter containing various elements able to bind transcription factors.

The inducible promoter is typically activated once the modified cell reaches the tumor.

Preferably, according to a first alternative, the sequence coding for IL-2 is inserted in a lentivector, preferably pCDH1, under control of a constitutive promoter. This alternative does not allow the control of IL-2 expression.

Preferably, according to a second alternative, the sequence coding for IL-2 is inserted in a lentivector, preferably pCDH1, under control of an inducible promoter.

The invention also relates to a modified cell expressing at least one cytokine, preferably at least one interleukin chosen from IL-10, IL-15, IL-13, IL-7A, IFNalpha, IFNbeta, IFNlambda, IFNgamma, IL-1A, IL-1B, IL-12 and IL-21, wherein the cell is chosen from myeloid cells, induced pluripotent stem cells (iPS) and hematopoietic stem cells (HSC), and wherein the cell comprises a first vector comprising a sequence coding for said interleukin under the control of an inducible or constitutive promoter, and optionally a second vector comprising a sequence coding for a chimeric antigen receptor (CAR),
wherein said CAR comprises:
   an extracellular antigen-binding domain which binds to a tumor antigen or an antigen present on cells of the tumor microenvironment (TME);
   optionally a hinge domain;
   a transmembrane domain, and
   an intracellular signaling domain.

All the above embodiments for the first vector are also applicable to said modified cell expressing a cytokine. In this case, the sequence of the cytokine to be expressed is introduced into the first vector instead of the sequence coding for IL-2.

Preferably, all the above embodiments for the first vector are also applicable to said modified cell expressing an interleukin (different from IL-2). In this case, the sequence of the interleukin to be expressed is introduced into the first vector instead of the sequence coding for IL-2.

Preferably, the modified myeloid cell, iPS or HSC according to the invention comprises a first vector comprising a sequence coding for IL-2 under the control of an inducible or constitutive promoter, and further a second vector comprising a sequence coding for a chimeric antigen receptor (CAR),
wherein said CAR comprises:
   an extracellular antigen-binding domain which binds to a tumor antigen or an antigen present on cells of the tumor microenvironment (TME);
   optionally a hinge domain;
   a transmembrane domain, and
   an intracellular signaling domain.

Such a cell is able to bind to tumor antigen or an antigen present on cells of the tumor microenvironment and typically presents a targeted effector activity such as an antigen-dependent phagocytosis of tumor cells.

Second Vector with Chimeric Antigen Receptor (CAR)

The modified myeloid cell, the modified iPS or the modified HSC of the invention preferably further comprises a second vector comprising a sequence coding for a chimeric antigen receptor (CAR). The presence of both said first vector and said second vector is called “CAR+2bands”.

In another embodiment, the CAR with its promoter can be included in the first vector (i.e. the vector comprising at least one interleukin sequence). In such a case, the modified myeloid cell, the modified iPS or the modified HSC of the invention only comprises the first vector, which encodes both for the interleukin and for the CAR; said vector is called “CAR2bands”. The CAR with its promoter can be upstream or downstream the at least one interleukin sequence. Preferably, the CAR with its promoter is downstream the at least one interleukin sequence. Preferably, the CAR with its promoter is downstream the IL2 sequence. Preferably, the CAR2bands vector comprises the nucleic acid sequence SEQ ID NO:9. Thus, according to this embodiment, preferably the modified myeloid cell, iPS or HSC according to the invention comprises a first vector (“CAR2bands”) comprising a sequence coding for IL-2 under the control of an inducible or constitutive promoter, and a sequence coding for a chimeric antigen receptor (CAR) under the control of an inducible or constitutive promoter,
wherein said CAR comprises:
   an extracellular antigen-binding domain which binds to a tumor antigen or an antigen present on cells of the tumor microenvironment (TME);
   optionally a hinge domain;
   a transmembrane domain, and
   an intracellular signaling domain.
      Preferably, the CAR2bands comprises the CAR with its promoter downstream the sequence coding for IL-2 under the control of an inducible or constitutive promoter. In other words, preferably, the CAR2bands comprises the CAR with its promoter in 3′ of the sequence coding for IL-2 under the control of an inducible or constitutive promoter.

The CAR of the invention comprises, from its N-terminal end to its C-terminal end:
an extracellular antigen-binding domain which binds to a tumor antigen or an antigen present on cells of the TME;
optionally a hinge domain;
a transmembrane domain; and
an intracellular signaling domain.

Between each domain, a linker, identical or different, may be present. Preferably, the CAR does not comprise any linker between the different domains. In other words, the CAR is obtained by direct fusion of the different domains.

By “antigen-binding domain”, it is meant any polypeptide or fragment thereof, such as an antibody fragment variable domain, either naturally-derived or synthetic, which binds to an antigen. Antigen-binding domains notably include polypeptides derived from antibodies, such as single chain variable fragments (scFv), Fab, Fab′, F(ab′)2, Fv fragments and nanobodies; polypeptides derived from T cell receptors (TCR), such as TCR variable domains; and any ligand or receptor fragment that binds to the antigen. Said antigen-binding domain has antigen specificity for a tumor antigen or a TME antigen. An «antigen-binding domain which has antigen specificity for a tumor antigen” is an antigen-binding domain that binds to an antigen on a tumor. An «antigen-binding domain which has antigen specificity for a TME antigen” is an antigen-binding domain that binds to an antigen which is present on cells of the tumor microenvironement (TME). The TME includes the tissues and cells around a tumor; it notably includes the surrounding blood vessels, immune cells such as Treg cells or immunosuppressive macrophages, fibroblasts, signaling molecules and the extracellular matrix.

Preferably, the tumor antigen is chosen from antigens expressed at the surface of tumor cells at higher levels than on other cell types. Preferably, the tumor antigen is chosen from CD19, MUC16, MUC1, CA1X, carcinoembryonic antigen (CEA), CD8, CD7, CD 10, CD20, CD22, CD30, CLL1, CD33, CD34, CD38, CD41, CD44, CD49f, CD56, CD74, CD133, CD138, EGP-2, EGP-40, EpCAM, erb-B2,3,4, FBP, Fetal acetylcholine receptor, folate receptor-a, GD2, GD2Ac, GD3, ITER-2, hTERT, IL-13R-a2, K-light chain, KDR, LeY, LI cell adhesion molecule, MAGE-A1, Mesothelin, ERBB2, MAGEA3, p53, MARTI, GP100, Proteinase 3 (PR1), Tyrosinase, Survivin, EphA2, NKG2D ligands, NY-ESO-1, oncofetal antigen (h5T4), PSCA, PSMA, ROR1, TAG-72, VEGF-R2, WT-I, BCMA, CD123, CD44V6, NKCS1, EGF1R, EGFR-VIII, CD99, CD70, ADGRE2, CCR1, LILRB2, PRAME, CCR4, CD5, CD3, TRBC1, TRBC2, TIM-3, Integrin B7, ICAM-1, CD70, Tim3, CLEC12A, ER, human telomerase reverse transcriptase (hTERT), mouse double minute 2 homolog (MDM2), cytochrome P450 1B1 (CYP1B), HER2/neu, p95HER2, Wilms' tumor gene 1 (WT1), livin, alphafetoprotein (AFP), prostate-specific membrane antigen (PSMA), cyclin (DI), mesothelin, B-cell maturation antigen (BCMA) and tumor-associated calcium signal transducer 2 (TROP2).

Preferably, the TME antigen is chosen from antigens expressed by activated CAF such as FAP (Fibroblast Activation Protein), antigens expressed by T regs and antigens expressed by protumoral myeloid cells such as TREM-2. Preferably, the TME antigen is chosen from FAP, antigens expressed by Tregs and TREM-2.

Preferably, the tumor antigen or TME antigen is CD19. More preferably, the extracellular antigen-binding domain which binds to a tumor antigen or a TME antigen is an anti-CD19 binding domain, preferably an anti-CD19 scFV.

Preferably, the extracellular antigen-binding domain comprises the amino acid sequence:

(SEQ ID NO: 3)
MALPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGGSGGGGSGGGGSEVKLQES
GPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAM
DYWGQGTSVTVSS.

By “hinge domain”, it is meant any hinge domain present in immunoglobulins or in CD molecules.

Preferably, the hinge domain is the one of CD8. CD8 comprises an alpha chain (CD8a) and a beta chain (CD8b). Preferably, the hinge domain is the one of the CD8a chain.

The human version of CD8a may be found in Uniprot under accession number Q8TAW8. CD8a comprises 235 amino acids. The hinge domain is the fragment of amino acids 138 to 182 of said sequence, which corresponds to SEQ ID NO:4.

Preferably, the hinge domain is the one of CD8a, preferably of human CD8a.

Preferably, the hinge domain comprises the amino acid sequence

(SEQ ID NO: 4)
TTTPAPRPPTPAPTIASQPLSLRPEACRPAAGGAVHTRGLDFACD.

By “transmembrane domain”, it is meant a single-pass or a multipass transmembrane sequence.

Single-pass transmembrane regions are found in certain CD molecules, tyrosine kinase receptors, serine/threonine kinase receptors, TGF, BMP, activin and phosphatases. Single-pass transmembrane regions often include a signal peptide region and a transmembrane region of about 20 to about 25 amino acids, many of which are hydrophobic amino acids and can form an alpha helix. A short track of positively charged amino acids often follows the transmembrane span to anchor the protein in the membrane.

Multipass transmembrane domains are present in proteins such as ion pumps, ion channels and transporters, and include two or more helices that span the membrane multiple times.

Sequences for single-pass and multipass transmembrane domains are known and can be selected for incorporation into the CAR.

The transmembrane domain can be chosen from wild-type transmembrane domains and mutated transmembrane domains. Mutated transmembrane domains may be modified by a mutation, such as an amino acid substitution (for example, an amino acid which is typically charged is substituted by a hydrophobic residue). Preferably, the transmembrane domain is the one of the alpha, beta or zeta chain of the T cell receptor, CD3-8, CD3zeta, CD4, CD5, CD8, CD8a, CD9, CD16, CD22, CD28, CD33, CD38, CD64, CD80, CD86, CD134, CD137 or CD154. Preferably, the transmembrane domain is a CD8 transmembrane domain.

The transmembrane domain may also be synthesized de novo, comprising mostly hydrophobic residues, such as, for example, leucine and valine.

According to the invention, the transmembrane domain is fused at its N-terminal end to the extracellular antigen-binding domain of the CAR, and at its C-terminal end to the intracellular signaling domain.

In certain embodiments, a short polypeptide linker may form the linkage between the transmembrane domain and the intracellular signaling domain of the CAR.

The CAR may further comprise a stalk, that is, an extracellular region of amino acids between the extracellular antigen-binding domain and the transmembrane domain. For example, the stalk may be a sequence of amino acids naturally associated with the selected transmembrane domain.

Preferably, the CAR comprises a CD8 transmembrane domain. Preferably, the CAR comprises a CD8 transmembrane domain, and a CD8 hinge domain. Said hinge domain is preferably fused (preferably directly), at its C-terminal end, to the N-terminal end of the transmembrane domain.

Preferably, the transmembrane domain comprises the amino acid sequence IYIWAPLAGTCGVLLLSLVITLYC (SEQ ID NO:5). This transmembrane domain is the one of human CD8.

Preferably, the hinge domain comprises the amino acid sequence

(SEQ ID NO: 4)
TTTPAPRPPTPAPTIASQPLSLRPEACRPAAGGAVHTRGLDFACD.

By “intracellular domain”, it is meant an intracellular signaling domain at the C-terminal end of the CAR. This intracellular domain can be chosen depending on the targeted effector activity aimed by the CAR. The intracellular domain can be derived from other receptors and can be designed to elicit a given cell function, such as phagocytosis, inflammatory activation or TME modulation.

The intracellular domain can comprise one or more intracellular signaling domains derived from a phagocytic receptor, a scavenger receptor or an integrin receptor. For example, the intracellular domain can comprise one or more intracellular signaling domains that promote phagocytic activity, an inflammatory response or integrin activation.

The intracellular signaling domain may be derived from a phagocytic or tethering receptor or may comprise a phagocytosis activation domain. Preferably, the intracellular signaling domain that promote phagocytic activity (i.e. also called phagocytosis activation domain) comprises an intracellular signaling domain derived from FcγR, FcαR or FcεR. In some embodiments, the intracellular signaling domain is derived from a receptor other than a phagocytic receptor selected from Megf10, MerTk, FcR-alpha or Bai1. In some embodiments, the intracellular signaling domain is derived from a phagocytic receptor chosen from lectin, dectin 1, CD206, scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fc-alpha receptor I, CR1, CD35, CR3, CR4, Tim-1, Tim-4 and CD169.

Preferably, the intracellular signaling domains that promote an inflammatory response (i.e. also called proinflammatory signaling domain) comprises a PI3-kinase (PI3K) recruitment domain. Preferably, it comprises an intracellular signaling domain of TLR3, TLR4, TLR9, MYD88, TRIF, RIG-1, MDA5, IFN receptor, NLRP-1, NLRP-2, NLRP-3, NLRP-4, NLRP-5, NLRP-6, NLRP-7, NLRP-8, NLRP-9, NLRP-10, NLRP-11, NLRP-12, NLRP-13, NLRP-14, NOD1, NOD2, Pyrin, AIM2, NLRC4 and/or CD40.

Preferably, the intracellular domain comprises at least two intracellular signaling domains, which comprise:
either (i) a first intracellular signaling domain derived from FcγR or FcεR, and (ii) a second intracellular signaling domain: (A) comprising a PI3K recruitment domain, or (B) derived from CD40;
or (i) a first intracellular signaling domain derived from a phagocytic receptor, and (ii) a second intracellular signaling domain: (A) comprising a PI3K recruitment domain, or (B) derived from CD40.

Examples of the intracellular domain include a fragment or domain from one or more molecules or receptors including, but are not limited to, STING, cGAS, TCR, CD3 zeta, CD3 gamma, CD3 delta, CD3 epsilon, CD86, CD79a, CD79b, DAP10, DAP 12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD I id, ITGAE, CD103, ITGAL, CDI la, LFA-1, ITGAM, CDI lb, ITGAX, CDI lc, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGLI, CD100 (SEMA4D), CD69, SLAMF6 (NTB-A, Ly108), SLAM (SLAMFi, CD150, IPO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8 or TLR9.

Preferably, the intracellular signaling domain of the invention comprises, from N-terminal to C-terminal, a first intracellular signaling domain comprising the CD40 cytotail (cytoplasmic tail), which is fused to a second intracellular signaling domain comprising the CD3zeta intracellular domain.

By “CD40 cytotail”, it is meant the cytosolic domain of the CD40 molecule. CD40, also called TNFRSF5, is a costimulatory protein found on antigen-presenting cells, and is required for their activation. The sequence of human CD40 (hCD40) may be found in Uniprot under accession number P25942. It comprises 277 amino acids. The fragment comprising amino acids 216-277 of said sequence is the cytosolic part. Said fragment corresponds to SEQ ID NO:6.

Preferably, the intracellular signaling domain comprises a CD40 cytotail which is a fragment of human CD40.

Preferably, the intracellular signaling domain comprises the amino acid sequence

(SEQ ID NO: 6)
KKVAKKPTNKAPHPKQEPQEINFPDDLPGSNTAAPVQETLHGCQPVTQED
GKESRISVQERQ.

Said first intracellular signaling domain is fused, at its C-terminal end, to a second intracellular signaling domain comprising the CD3zeta intracellular domain. Preferably said fusion is directly performed, i.e. without any linker.

CD3zeta, also called OKT3 or CD247, is a member of the T cell receptor (TCR) complex. In humans, in 95% of T cells the TCR consists of an alpha chain and a beta chain, whereas in 5% of T cells the TCR consists of gamma and delta chains. In the plasma membrane, the TCR chains alpha and beta associate with six additional adaptor proteins to form an octameric complex. Said complex comprises both alpha and beta chains, forming the ligand-binding site, but also one CD3gamma chain, one CD3delta chain, two CD3epsilon chains and two CD3zeta chains.

The sequence of human CD3zeta chain (hCD3zeta) may be found in Uniprot under accession number P20963. It comprises 164 amino acids. The fragment comprising amino acids 52-164 of said sequence is the cytosolic part. Said fragment corresponds to SEQ ID NO:7.

Preferably, the second intracellular signaling domain comprises the human CD3zeta intracellular domain.

Preferably, the second intracellular signaling domain comprises the amino sequence

(SEQ ID NO: 7)
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR.

Preferably, the intracellular signaling domain is either a first intracellular signaling domain comprising the CD40 cytoplasmic tail, preferably of sequence SEQ ID NO:6, or a first intracellular signaling domain comprising the CD40 cytoplasmic tail, preferably of sequence SEQ ID NO:6, which is fused to a second intracellular signaling domain comprising the CD3zeta intracellular domain, preferably of sequence SEQ ID NO:7.

Preferably, the modified cell of the invention is a myeloid cell and comprises a first vector comprising a sequence coding for IL-2 under the control of an inducible or constitutive promoter, and a second vector comprising a sequence coding for a chimeric antigen receptor (CAR),
wherein said CAR comprises:
   an extracellular antigen-binding domain which has antigen specificity for a tumor antigen or a TME antigen;
   optionally a hinge domain,
   a transmembrane domain; and
   an intracellular signaling domain comprising STING or one of its fragments.

Said cell is called “modified myeloid cell with a CAR including STING or one of its fragments” in the present application.

The above embodiments and definitions for the modified myeloid cell expressing a CAR, except the intracellular signaling domain, also apply to the modified myeloid cell with a CAR including STING or one of its fragments.

Preferably the modified myeloid cell according to the invention comprises a first vector (“CAR2bands”) comprising a sequence coding for IL-2 under the control of an inducible or constitutive promoter, and a sequence coding for a chimeric antigen receptor (CAR) under the control of an inducible or constitutive promoter,
wherein said CAR comprises:
   an extracellular antigen-binding domain which binds to a tumor antigen or an antigen present on cells of the tumor microenvironment (TME);
   optionally a hinge domain;
   a transmembrane domain, and
   an intracellular signaling domain comprising STING or one of its fragments.

Preferably, the CAR2bands comprises the CAR with its promoter downstream the sequence coding for IL-2 under the control of an inducible or constitutive promoter. In other words, preferably, the CAR2bands comprises the CAR with its promoter in 3′ of the sequence coding for IL-2 under the control of an inducible or constitutive promoter.

The intracellular signaling domain of the modified myeloid cell with a CAR including STING or one of its fragments, comprises STING or one of its fragments.

The STimulator of INterferon Genes (STING) protein is an endoplasmic reticulum (ER) resident protein that plays a central role in innate immunity. Indeed, STING is an adaptor protein that orchestrates transcriptional activation of type I interferons and inflammatory cytokines in the presence of pathological nucleic acid species. STING activation relies on the detection of dsDNA, ssDNA, or RNA:DNA hybrids by the cyclic GMP-AMP synthetase (cGAS) pathogen recognition receptor. Association of cGAS with these nucleic acid species in the cytosol was found to lead to cGAS-dependent synthesis of cyclic GMP-AMP (cGAMP). Interaction of cGAMP with STING activates a pathway that finally leads to the transcription of pro-inflammatory cytokines and type I interferons.

The sequence of human STING may be found in Uniprot under accession number A0A2R3XZB7. It comprises 379 amino acids. Preferably, a sequence with some amino acid deletions in the N-terminal end is used. STING may be used in its wild-type version, or in a mutated form to attenuate its activity.

Preferably, a fragment of STING is used, preferably comprising deletions of the N-terminal end. Preferably, the fragment corresponds to amino acids 137 to 379 of A0A2R3XZB7.

Preferably, the intracellular signaling domain comprises the amino sequence

(SEQ ID NO: 8)
KGLAPAEISAVCEKGNFNVAHGLAWSYYIGYLRLILPELQARIRTYNQHY
NNLLRGAVSQRLYILLPLDCGVPDNLSMADPNIRFLDKLPQQTGDRAGIK
DRVYSNSIYELLENGQRAGTCVLEYATPLQTLFAMSQYSQAGFSREDRLE
QAKLFCRTLEDILADAPESQNNCRLIAYQEPADDSSFSLSQEVLRHLRQE
EKEEVTVGSLKTSAVPSTSTMSQEPELLISGMEKPLPLRTDFS.

Preferably, the intracellular signaling domain is encoded by the nucleic sequence SEQ ID NO:10.

The present invention further relates to a modified cell comprising a CAR, wherein said CAR comprises:
an extracellular antigen-binding domain which has antigen specificity for a tumor antigen or a TME antigen;
a transmembrane domain; and
an intracellular signaling domain comprising a chimeric fragment of STING;
and wherein said modified cell is a myeloid cell.

The above embodiments and above definitions for the CAR myeloid cell, except the intracellular signaling domain, are also valid for such a modified myeloid cell with a CAR including a chimeric fragment of STING.

By “a chimeric fragment of STING”, it is meant a modified STING C-terminal tail (CTT). Said modified STING CTT contains two sequence motifs known as IRF3 and TBK1 and, optionally, an additional fish-specific NF-KB motif. Preferably, said modified STING CTT is a chimera construct where IRF3 motif is from human STING, and TBK1 and NF-KB motifs are from human STING or fish STING. Preferably, the fish STING is a zebrafish STING. Preferably, said modified STING CTT is a chimera construct where IRF3 and TBK1 motifs are from human STING and the NF-KB is from zebrafish STING (said CAR is called “CAR-STINGtz”). Preferably, CAR-STINGtz comprises the amino sequence:

(SEQ ID NO: 11)
KGLAPAEISAVCEKGNFNVAHGLAWSYYIGYLRLILPELQARIRTYNQHY
NNLLRGAVSQRLYILLPLDCGVPDNLSMADPNIRFLDKLPQQTGDRAGIK
DRVYSNSIYELLENGQRAGTCVLEYATPLQTLFAMSQYSQAGFSREDRLE
QAKLFCRTLEDILADAPESQNNCRLIAYQEPADDSSFSLSQEVLRHLRQE
EKEEVTVGSLKTSAVPSTSTMSQEPELLISGMEKPLPLRTDPVETTDYFN
PSSAMKQN.

Preferably, CAR-STINGtz is encoded by the following nucleic sequence:

(SEQ ID NO: 12)
AAGGGCCTGGCCCCAGCTGAGATCTCTGCAGTGTGTGAAAAAGGGAATTT
CAACGTGGCCCATGGGCTGGCATGGTCATATTACATCGGATATCTGCGGC
TGATCCTGCCAGAGCTCCAGGCCCGGATTCGAACTTACAATCAGCATTAC
AACAACCTGCTACGGGGTGCAGTGAGCCAGCGGCTGTATATTCTCCTCCC
ATTGGACTGTGGGGTGCCTGATAACCTGAGTATGGCTGACCCCAACATTC
GCTTCCTGGATAAACTGCCCCAGCAGACCGGTGACCGTGCTGGCATCAAG
GATCGGGTTTACAGCAACAGCATCTATGAGCTTCTGGAGAACGGGCAGCG
GGCGGGCACCTGTGTCCTGGAGTACGCCACCCCCTTGCAGACTTTGTTTG
CCATGTCACAATACAGTCAAGCTGGCTTTAGCCGGGAGGATAGGCTTGAG
CAGGCCAAACTCTTCTGCCGGACACTTGAGGACATCCTGGCAGATGCCCC
TGAGTCTCAGAACAACTGCCGCCTCATTGCCTACCAGGAACCTGCAGATG
ACAGCAGCTTCTCGCTGTCCCAGGAGGTTCTCCGGCACCTGCGGCAGGAG
GAAAAGGAAGAGGTTACTGTGGGCAGCTTGAAGACCTCAGCGGTGCCCAG
TACCTCCACGATGTCCCAAGAGCCTGAGCTCCTCATCAGTGGAATGGAAA
AGCCCCTCCCTCTCCGCACGGATCCTGTGGAGACCACCGATTATTTTAAC
CCATCTAGCGCAATGAAACAAAACTAA.

Said intracellular signaling domain comprising STING or one of its fragments may further comprise the CD40 cytotail, preferably as described above, and/or the CD3zeta intracellular domain, preferably as described above.

Preferably, the CAR comprises, from its N-terminal end to its C-terminal end:
an extracellular antigen-binding domain of sequence SEQ ID NO:3,
optionally a hinge domain of sequence SEQ ID NO:4,
a transmembrane domain of sequence SEQ ID NO:5,
either a first intracellular signaling domain of sequence SEQ ID NO:6, or a first intracellular signaling domain of sequence SEQ ID NO:6 which is fused, preferably directly, to a second intracellular signaling domain of sequence SEQ ID NO:7.

Preferably, the CAR comprises, from its N-terminal end to its C-terminal end:
an extracellular antigen-binding domain of sequence SEQ ID NO:3,
optionally a hinge domain of sequence SEQ ID NO:4,
a transmembrane domain of sequence SEQ ID NO:5,
a first intracellular signaling domain of sequence SEQ ID NO:6, fused, preferably directly, to a second intracellular signaling domain of sequence SEQ ID NO:7.

Preferably, the CAR comprises, from its N-terminal end to its C-terminal end:
an extracellular antigen-binding domain of sequence SEQ ID NO:3,
optionally a hinge domain of sequence SEQ ID NO:4,
a transmembrane domain of sequence SEQ ID NO:5, and
an intracellular signaling domain of sequence SEQ ID NO:11.

The modified myeloid cell, iPS or HSC according to the invention, comprising the first and second vectors, preferably presents targeted effector activity. By “targeted effector activity”, it is meant at least one effector activity chosen from phagocytosis, targeted cellular cytotoxicity, production of cytokines, production of reactive oxygen species (ROS), myeloid activation, antigen processing and presentation to T cells, and in vivo capacity in NSG mice complemented with human T cells to induce human antigen-dependent tumor regression. Preferably, the targeted effector activity is selected from antigen-dependent phagocytosis of tumor cells, antigen-dependent tumor cell cytokine secretion, and in vivo capacity in NSG mice complemented with human T cells to induce human antigen-dependent tumor regression. Said human antigen-dependent tumor regression is probably mediated by both macrophage phagocytosis of tumor cells and tumor specific T cell killing of tumor cells. Antigen-dependent phagocytosis of tumor cells and antigen-dependent tumor cell cytokine secretion may be measured according to methods well-known in the art, which are illustrated in the examples. In vivo capacity in NSG mice complemented with human T cells to induce human antigen-dependent tumor regression is evaluated according to the protocol described in the examples.

All the above embodiments for the second vector are also applicable to the modified cell expressing a cytokine.

All the above embodiments for the second vector are also applicable to the modified cell expressing an interleukin (different from IL-2). In such a case, the sequence of the interleukin to be expressed is introduced into the first vector instead of the sequence coding for IL-2.

In another embodiment, the invention also relates to a modified cell expressing at least one cytokine, preferably an interleukin, wherein the cell is chosen from myeloid cells, induced pluripotent stem cells (iPS) and hematopoietic stem cells (HSC), and wherein the cell comprises a first vector comprising a sequence coding for said cytokine under the control of an inducible or constitutive promoter, and optionally a second vector comprising a sequence coding for a CAR, wherein said CAR comprises at least the CD40 cytotail as intracellular signaling domain. In this embodiment, said modified cell is preferably a macrophage.

In another embodiment, the invention also relates to a modified cell expressing at least one interleukin chosen from IL-2, IL-10, IL-15, IL-13, IL-7A, IFNalpha, IFNbeta, IFNlambda, IFNgamma, IL-1A, IL-1B, IL-12 and IL-21, wherein the cell is chosen from myeloid cells, induced pluripotent stem cells (iPS) and hematopoietic stem cells (HSC), and wherein the cell comprises a first vector comprising a sequence coding for said interleukin under the control of an inducible or constitutive promoter, and optionally a second vector comprising a sequence coding for a CAR, wherein said CAR comprises at least the CD40 cytotail as intracellular signaling domain. In this embodiment, said modified cell is preferably a macrophage.

The present invention also relates to the nucleic acid sequence coding for a CAR according to the invention. Said nucleic acid sequence may be a DNA or RNA sequence. Said nucleic acid sequence may be used in therapy, especially for treating a cancer, an autoimmune disease or an inflammatory disease. Preferably, said nucleic acid sequence is administered to a subject, preferably by injection. Accordingly, the macrophages of said subject receive said nucleic acid sequence, and subsequently express the CAR, notably the CAR including STING or one of its fragments, preferably the CAR-STINGtz.

Preferably, the nucleic sequence coding for the intracellular domain of said CAR-STINGtz is the sequence SEQ ID NO:12. Preferably, the nucleic sequence comprises the sequence SEQ ID NO:12.

### Therapeutic Uses

The present invention also relates to the use of a modified myeloid cell, a modified iPS or a modified HSC according to the invention, as a medicament.

The present invention also relates to a pharmaceutical composition comprising a modified myeloid cell, a modified iPS or a modified HSC according to the invention, and a pharmaceutical acceptable carrier.

The present invention also relates to the use of the modified myeloid cell, the modified iPS or the modified HSC according to the invention, or of the pharmaceutical composition described above, in the treatment of cancer, an autoimmune disease or an inflammatory disease. The inflammatory disease may be an autoimmune disease.

All these embodiments also apply to the modified cell expressing a cytokine.

All these embodiments also apply to the modified cell expressing an interleukin (different from IL-2). The sequence of the interleukin to be expressed in introduced into the first vector instead of the sequence coding for IL-2.

Preferably, the myeloid cells are obtained from a blood sample of the treated patient (treated donor). Preferably, they are modified and reinjected to the patient (donor), i.e. they are autologous.

By “autologous”, the present invention means that the modified myeloid cells or the therapeutic composition comprising the modified myeloid cells are used as a treatment for the patient from which the myeloid cells are originated.

The present invention also relates to products containing a modified myeloid cell, a modified iPS or a modified HSC according to the invention, and a CAR-T cell, as a combined preparation for simultaneous, separate or sequential use in treatment of cancer, an autoimmune disease or an inflammatory disease.

CAR-T cells are well-known in the art. Preferably, CAR-T cells are chosen from tisagenlecleucel, axicabtagene ciloleucel, brexucabtagene autoleucel, lisocabtagene maraleucel and idecabtagene vicleucel.

The present invention also relates to products containing a modified myeloid cell, a modified iPS or a modified HSC according to the invention, and an Immune Checkpoint Inhibitor (ICI) as a combined preparation for simultaneous, separate or sequential use in treatment of cancer, an autoimmune disease or an inflammatory disease.

An “immune checkpoint inhibitor” refers to any compound inhibiting the function of an immune checkpoint protein. Inhibition includes reduction of function and full blockade. In particular, the immune checkpoint protein is a human immune checkpoint protein. Thus the immune checkpoint protein inhibitor is preferably an inhibitor of a human immune checkpoint protein.

Immune checkpoint proteins that may be quoted are CTLA-4, PD-1, PD-L1, PD-L2, LAG-3, BTLA, B7H3, B7H4, TIM3, KIR (such as KIR3DL2, KIR2DL1/2/3, KIR2L3), TIGIT, VISTA, IDO, CEACAM-1 or A2aR.

The immune checkpoint inhibitors may be drugs such as small molecules, recombinant forms of ligand or receptors, or preferably antibodies, such as human antibodies. Known inhibitors of the immune checkpoint proteins or analogs thereof may be used, in particular chimerized, humanized or human forms of antibodies.

Preferably the ICI is selected from an inhibitor of CTLA-4, PD-1, PD-L1, PD-L2, LAG-3, BTLA, B7H3, B7H4, TIM3, KIR (such as KIR3DL2, KIR2DL1/2/3, KIR2L3), TIGIT, VISTA, IDO, CEACAM-1 or A2aR. Preferably, the ICI is an anti-CTLA-4 antibody, more preferably tremelimumab or ipilimumab. In certain aspects, the ICI is an anti-killer-cell immunoglobulin-like receptor (KIR) antibody, more preferably lirilumab and IPH4102. Preferably, the ICI is an anti-PD-1 antibody, more preferably chosen from nivolumab (ONO-4538, BMS-936558, MDX1106, GTPL7335 or Opdivo), pembrolizumab (MK-3475, MK03475, lambrolizumab, SCH-900475 or Keytruda), pidilizumab, AMP-514, cemiplimab (REGN2810), CT-011, BMS 936559, MPDL3280A, AMP-224, tislelizumab (BGB-A317), spartalizumab (PDR001 or PDR-001), ABBV-181, JNJ-63723283, BI 754091, MAG012, TSR-042, AGEN2034 and antibodies described in International patent applications WO2004004771, WO2004056875, WO2006121168, WO2008156712, WO2009014708, WO2009114335, WO2013043569 and WO2014047350. Preferably, the inhibitor of PD-L1 is durvalumab, atezolizumab, LY3300054 or avelumab. Preferably, the inhibitor of PD-L2 is rHlgM12B7. Preferably, the LAG3 inhibitor is IMP321, BMS-986016 or inhibitors of the LAG3 receptor described in US patent U.S. Pat. No. 5,773,578. Preferably, the inhibitor of A2aR is PBF-509. Preferably, the inhibitor of CTLA-4 is an anti-CTLA-4 antibodies including, but not limited to, ipilimumab (see, e.g., US patents U.S. Pat. Nos. 6,984,720 and 8,017,114), tremelimumab (see, e.g., US patents U.S. Pat. Nos. 7,109,003 and 8,143,379), single chain anti-CTLA4 antibodies (see, e.g., International patent applications WO1997020574 and WO2007123737) and antibodies described in US patent U.S. Pat. No. 8,491,895. Example of anti-VISTA antibodies are described in US patent application US20130177557. Preferably, the ICI is chosen from tremelimumab, ipilimumab, lirilumab, nivolumab, pembrolizumab, pidilizumab, AMP-514, REGN2810, CT-011, BMS 936559, MPDL3280A, AMP-224, durvalumab, atezolizumab, avelumab, rHIgM12B7, IMP321, BMS-986016 and PBF-509.

The present invention also relates to products containing the modified myeloid cell, the modified iPS or the modified HSC according to the invention, and an immune checkpoint therapy related to co-stimulatory antibodies delivering positive signals through immune-regulatory receptors including but not limited to ICOS, CD137, CD27, OX-40 and GITR, as a combined preparation for simultaneous, separate or sequential use in treatment of cancer, an autoimmune disease or an inflammatory disease.

The present invention also relates to products containing the modified myeloid cell, the modified iPS or the modified HSC according to the invention, and additional cancer therapies as a combined preparation for simultaneous, separate or sequential use in treatment of cancer. In particular, products containing the modified myeloid cell, the modified iPS or the modified HSC according to the invention may be administered in combination with targeted therapy, immunotherapy such as immune checkpoint therapy and/or immune checkpoint inhibitor, co-stimulatory antibodies, chemotherapy and/or radiotherapy.

In some embodiments, the products containing the modified myeloid cell, the modified iPS or the modified HSC according to the invention may be used in combination with targeted therapy. As used herein, the term “targeted therapy” refers to targeted therapy agents, drugs designed to interfere with specific molecules necessary for tumor growth and progression. For example, targeted therapy agents such as therapeutic monoclonal antibodies target specific antigens found on the cell surface, such as transmembrane receptors or extracellular growth factors. Small molecules can penetrate the cell membrane to interact with targets inside a cell. Small molecules are usually designed to interfere with the enzymatic activity of the target protein such as for example proteasome inhibitor, tyrosine kinase or cyclin-dependent kinase inhibitor, histone deacetylase inhibitor. Targeted therapy may also use cytokines. Examples of such targeted therapy include: Ado-trastuzumab emtansine (HER2), Afatinib (EGFR (HER1/ERBB1), HER2), Aldesleukin (Proleukin), alectinib (ALK), Alemtuzumab (CD52), axitinib (kit, PDGFRbeta, VEGFR1/2/3), Belimumab (BAFF), Belinostat (HDAC), Bevacizumab (VEGF ligand), Blinatumomab (CD19/CD3), bortezomib (proteasome), Brentuximab vedotin (CD30), bosutinib (ABL), brigatinib (ALK), cabozantinib (FLT3, KIT, MET, RET, VEGFR2), Canakinumab (IL-1 beta), carfilzomib (proteasome), ceritinib (ALK), Cetuximab (EGFR), cofimetinib (MEK), Crizotinib (ALK, MET, ROS1), Dabrafenib (BRAF), Daratumumab (CD38), Dasatinib (ABL), Denosumab (RANKL), Dinutuximab (B4GALNT1 (GD2)), Elotuzumab (SLAMF7), Enasidenib (IDH2), Erlotinib (EGFR), Everolimus (mTOR), Gefitinib (EGFR), Ibritumomab tiuxetan (CD20), Sonidegib (Smoothened), Sipuleucel-T, Siltuximab (IL-6), Sorafenib (VEGFR, PDGFR, KIT, RAF), (Tocilizumab (IL-6R), Temsirolimus (mTOR), Tofacitinib (JAK3), Trametinib (MEK), Tositumomab (CD20), Trastuzumab (HER2), Vandetanib (EGFR), Vemurafenib (BRAF), Venetoclax (BCL2), Vismodegib (PTCH, Smoothened), Vorinostat (HDAC), Ziv-aflibercept (PIGF, VEGFA/B), Olaparib (PARP inhibitor).

In some embodiments, the products containing the modified myeloid cell, the modified iPS or the modified HSC according to the invention may be used in combination with chemotherapy. As used herein, the term “antitumor chemotherapy” or “chemotherapy” has its general meaning in the art and refers to a cancer therapeutic treatment using chemical or biochemical substances, in particular using one or several antineoplastic agents or chemotherapeutic agents. Chemotherapeutic agents include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; methylhydrazine derivatives including N-methylhydrazine (MIH) and procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; anthracyclines, nitrosoureas, antimetabolites, epipodophylotoxins, enzymes such as L-asparaginase; anthracenediones; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide; and pharmaceutically acceptable salts, acids or derivatives of any of the above.

In some embodiments, the products containing the modified myeloid cell, the modified iPS or the modified HSC according to the invention is administered to the patient in combination with radiotherapy for simultaneous, separate or sequential use in treatment of cancer, an autoimmune disease or an inflammatory disease. Suitable examples of radiation therapies include external beam radiotherapy (such as superficial X-rays therapy, orthovoltage X-rays therapy, megavoltage X-rays therapy, radiosurgery, stereotactic radiation therapy, Fractionated stereotactic radiation therapy, cobalt therapy, electron therapy, fast neutron therapy, neutron-capture therapy, proton therapy, intensity modulated radiation therapy (IMRT), 3-dimensional conformal radiation therapy (3D-CRT) and the like); brachytherapy; unsealed source radiotherapy; tomotherapy; and the like. Gamma rays are another form of photons used in radiotherapy. Gamma rays are produced spontaneously as certain elements (such as radium, uranium, and cobalt 60) release radiation as they decompose, or decay. In some embodiments, radiotherapy may be proton radiotherapy or proton minibeam radiation therapy. Proton radiotherapy is an ultra-precise form of radiotherapy that uses proton beams (Prezado Y, Jouvion G, Guardiola C, Gonzalez W, Juchaux M, Bergs J, Nauraye C, Labiod D, De Marzi L, Pouzoulet F, Patriarca A, Dendale R. Tumor Control in RG2 Glioma-Bearing Rats: A Comparison Between Proton Minibeam Therapy and Standard Proton Therapy. Int J Radiat Oncol Biol Phys. 2019 Jun. 1; 104(2):266-271. doi: 10.1016/j.ijrobp.2019.01.080; Prezado Y, Jouvion G, Patriarca A, Nauraye C, Guardiola C, Juchaux M, Lamirault C, Labiod D, Jourdain L, Sebrie C, Dendale R, Gonzalez W, Pouzoulet F. Proton minibeam radiation therapy widens the therapeutic index for high-grade gliomas. Sci Rep. 2018 Nov. 7; 8(1):16479. doi: 10.1038/s41598-018-34796-8). Radiotherapy may also be FLASH radiotherapy (FLASH-RT) or FLASH proton irradiation. FLASH radiotherapy involves the ultra-fast delivery of radiation treatment at dose rates several orders of magnitude greater than those currently in routine clinical practice (ultra-high dose rate) (Favaudon V, Fouillade C, Vozenin M C. The radiotherapy FLASH to save healthy tissues. Med Sci (Paris) 2015; 31: 121-123. DOI: 10.1051/medsci/20153102002); Patriarca A., Fouillade C. M., Martin F., Pouzoulet F., Nauraye C., et al. Experimental set-up for FLASH proton irradiation of small animals using a clinical system. Int J Radiat Oncol Biol Phys, 102 (2018), pp. 619-626. doi: 10.1016/j.ijrobp.2018.06.403. Epub 2018 Jul. 11).

It is also described a method for treating cancer, an autoimmune disease or an inflammatory disease in a subject in need thereof, comprising a step of administering to said subject a therapeutically effective amount of the modified myeloid cell, the modified PS or the modified HSC according to the invention.

It is also described a method for treating cancer, an autoimmune disease or an inflammatory disease, which comprises:
collecting myeloid cells from a patient;
modifying at least one of said myeloid cells by transducing said cell with a first vector comprising a sequence coding for IL-2 under the control of an inducible or constitutive promoter, and optionally with a second vector comprising a nucleic sequence coding for the CAR, preferably a lentiviral vector; and
re-injecting said modified myeloid cells into the patient.

By “cancer”, the invention means tumors. The tumors to be treated include primary tumors and metastatic tumors, as well as refractory tumors. Refractory tumors include tumors that fail to respond or are resistant to treatment with chemotherapeutic agents alone, antibodies alone, radiation alone or combinations thereof. Refractory tumors also encompass tumors that appear to be inhibited by treatment with such agents, but recur up to five years, sometimes up to ten years or longer after treatment is discontinued.

Examples of cancers that may be treated by the modified myeloid cell according to the invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extramammary paraganglioma, malignant; pheochromocytoma; glomangio sarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

Preferably, cancer is a solid tumor or a metastasis.

Examples of autoimmune diseases that may be treated by the modified myeloid cell according to the invention include, but are not limited to, rheumatoid arthritis, inflammatory bowel disease (Crohn disease, ulcerative colitis) or multiple sclerosis.

By “treatment” or “treat”, it is meant both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment.

By “therapeutic regimen” is meant the pattern of treatment of an illness, e.g., the pattern of dosing used during therapy. A therapeutic regimen may include an induction regimen and a maintenance regimen.

The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both.

The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

By a “therapeutically effective amount”, it is meant a sufficient amount of the modified myeloid cell, the modified PS or the modified HSC according to the invention, to treat the disease (e.g. cancer) at a reasonable benefit/risk ratio applicable to any medical treatment. It will be understood that the total daily usage of the product of the present invention will be decided by the attending physician within the scope of sound medical judgment. The specific therapeutically effective dose level for any particular patient will depend upon a variety of factors including the age, body weight, general health, sex and diet of the patient; the time of administration, route of administration, and rate of excretion of the specific compound employed; the duration of the treatment; drugs used in combination or coincidental with the product; and like factors well known in the medical arts. For example, it is well known within the skill of the art to start doses of the compound at levels lower than those required to achieve the desired therapeutic effect and to gradually increase the dosage until the desired effect is achieved.

By “pharmaceutical” or “pharmaceutically acceptable”, it is meant that molecular entities and compositions do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings.

Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The product can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The first and/or second vector, or the CAR2bands, may be administered via at least one lipid nanoparticle (LNP) or at least one liposome or at least one virus-like particle (VLP). LNPs refer to stable nucleic acid-lipid nanoparticles, particularly in the field of nucleic acid and mRNA drug delivery systems. Liposomes refer to spherical vesicles made of a lipid bilayer. The LNP or liposome comprise at least one ionizable lipid and at least one nucleic acid molecule. Preferably, the LNP or liposome further comprise at least one helper lipid. Preferably the helper lipid is chosen from phospholipids, cholesterol lipids, and polymers.

The phospholipid may typically be chosen from dioleoyl-phosphatidylethanolamine (DOPE) or a derivative thereof, distearoylphosphatidylcholine (DSPC) or a derivative thereof, distearoyl-phosphatidylethanolamine (DSPE) or a derivative thereof, stearoyloleoylphosphatidylcholine (SOPC) or a derivative thereof, 1-stearioyl-2-oleoyl-phosphatidyethanol amine (SOPE) or a derivative thereof, N-(2,3-dioleoyloxy)propyl)-N,N,N-trimethylammonium chloride (DOTAP) or a derivative thereof, or any combination thereof.

The cholesterol lipid may be cholesterol or a derivative thereof.

The polymer may be polyethylene glycol (PEG) or a derivative thereof.

The nucleic acid molecule is a DNA molecule or an RNA molecule. In some embodiments, the nucleic acid molecule is cDNA, mRNA, miRNA, siRNA, sgRNA, modified RNA, antagomir, antisense molecule, guide RNA molecule, CRISPR guide RNA molecule, peptide, therapeutic peptide, targeted nucleic acid, or any combination thereof.

In some embodiments, the liposome is a ligand-targeted liposome which surface is functionalized with at least one target ligand. Said target ligand may be chosen from antibodies, agonist peptides and aptamers. It allows a precise delivery of the first and/or second vector(s) to specific cells such as myeloid cells, preferably monocytes or macrophages, by recognizing corresponding receptors or antigens.

Virus-like particles (VLP) are vesicles having a hollow core and an envelope, that mimic viruses but that are not infectious. They can be made up of viral structural protein(s) that self-assemble into the virus-like structure, of chemical compound(s), or of polymers that are arranged in multiple layers surrounding a hollow core. Said VLP can package nucleic acid sequences corresponding to transcription units encoding the CAR and, optionally, transcription units encoding the at least one cytokine, preferably at least one interleukin.

The present invention also relates to a method of delivering at least one mRNA molecule encoding the first and/or second vector, or the CAR2bands to a subject in need thereof. In some embodiments, the LNP or the composition thereof delivers the mRNA molecule encoding the first and/or second vector, or the CAR2bands to a target, preferably myeloid cells. Said method may comprise a single administration or multiple administrations of the LNP or the composition thereof. In some embodiments, the LNP or the composition thereof is administered by a delivery route selected from the group consisting of intradermal, subcutaneous, intramuscular, intraventricular, intrathecal, oral delivery, intravenous, intratracheal, intraperitoneal, in utero delivery, or any combination thereof.

### Preparation Method

The present invention also relates to a method for manufacturing a modified myeloid cell, a modified PS or a modified HSC, wherein the method comprises:
providing at least one cell chosen from isolated myeloid cells, PS or HSC;
transducing said cell with at least a first vector, preferably a lentiviral vector, comprising a nucleic sequence coding for IL-2 or a nucleic sequence coding for at least one interleukin chosen from IL-10, IL-15, IL-13, IL-7A, IFNalpha, IFNbeta, IFNlambda, IFNgamma, IL-1A, IL-1B, IL-12 and IL-21, under the control of an inducible or constitutive promoter; and optionally, a second vector, preferably a lentiviral vector, comprising a sequence coding for a CAR.

Said CAR comprises:
an extracellular antigen-binding domain which binds to a tumor antigen or a TME antigen;
optionally a hinge domain;
a transmembrane domain; and
a first intracellular signaling domain. Preferably, the first intracellular signaling domain comprising the CD40 cytotail, which is fused to a second intracellular signaling domain comprising the CD3zeta intracellular domain.

The first step of the preparation method is the provision of at least one cell chosen from isolated myeloid cells, iPS or HSC.

Then, said cells are transduced with at least a first vector, preferably lentiviral vectors, comprising a sequence coding for IL-2 under the control of an inducible or constitutive promoter. The first vector may be used to introduce IL-2 into an isolated myeloid cell, preferably a monocyte.

Preferably, said cells are transduced with two vectors, preferably lentiviral vectors, the first one comprising a sequence coding for IL-2 under the control of an inducible or constitutive promoter, and the second one comprising a nucleic sequence coding for a CAR. The vectors may be used to introduce the CAR and IL-2 into an isolated myeloid cell, preferably a monocyte.

Alternatively, said cells may be transduced with a single vector (i.e. first vector) comprising a sequence coding for IL-2 under the control of an inducible or constitutive promoter, and a nucleic sequence coding for a CAR under the control of a promoter.

In one embodiment, the vector is a plasmid vector, a viral vector, a retrotransposon (e.g. piggyback, sleeping beauty) or a site directed insertion vector (e.g. CRISPR, Zn finger nucleases, TALEN). Preferably, the vector is a viral vector, preferably a lentiviral vector. Vectors, including those derived from retroviruses such as lentivirus, are suitable tools to achieve long-term gene transfer since they allow long-term, stable integration of a transgene and its propagation in daughter cells. Lentiviral vectors have the added advantage over vectors derived from onco-retroviruses, such as murine leukemia viruses, in that they can transduce non-proliferating cells. They also have the added advantage of resulting in low immunogenicity in the subject into which they are introduced.

The expression of natural or synthetic nucleic acids is typically achieved by operably linking a nucleic acid to a promoter, and incorporating the construct into an expression vector. The vector is one generally capable of replication in a mammalian cell, and/or also capable of integration into the cellular genome of the mammal. Typical vectors contain transcription and translation terminators, initiation sequences and promoters useful for regulation of the expression of the desired nucleic acid sequence.

The nucleic sequence (nucleic acid) coding for said CAR or said IL-2 can be cloned into any number of different types of vectors. For example, the nucleic acid can be cloned into a vector including, but not limited to a plasmid, a phagemid, a phage derivative, an animal virus or a cosmid. Vectors of particular interest include expression vectors, replication vectors, probe generation vectors and sequencing vectors.

The expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY). Viruses which are useful as vectors include retroviruses, adenoviruses, adeno-associated viruses, herpes viruses and lentiviruses. In general, a suitable vector contains an origin of replication functional in at least one organism, a promoter sequence, convenient restriction endonuclease sites, and one or more selectable markers. Additional promoter elements, e.g., enhancers, regulate the frequency of transcriptional initiation. Depending on the promoter, it appears that individual elements can function either cooperatively or independently to activate transcription.

In one embodiment, the vector is a recombinant lentiviral vector with modified tropism comprising (i) a mutant glycoprotein (G protein) which ablates the natural receptor tropism and (ii) an insertion of an antibody, a receptor ligand or a peptide targeting specifically a cell, preferably a myeloid cell, preferably a monocyte or a macrophage. Said G protein plays a critical role during the initial steps of virus infection as it is responsible for virus attachment to specific receptors. After binding, G triggers the fusion between the viral and endosomal membranes, which releases the viral genome in the cytosol for the subsequent steps of infection. Preferably, said recombinant lentiviral vector comprising a mutant G protein allows for an antigen-specific infection of myeloid cells, preferably of monocytes or macrophages.

In order to assess expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like. Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

The method comprises introducing into said cell a first vector, said vector comprising a sequence coding for IL-2 under the control of a cytokine specific promoter. Preferably, the cytokine specific promoter is chosen from promoter of interleukin-6 or the promoter of interleukin-8.

Preferably, the gene of IL-2 is a human gene of nucleic sequence SEQ ID NO:1.

Preferably, the method comprises introducing into said cell a second vector, said vector comprising a CAR. Preferably, the CAR comprises, from its N-terminal end to its C-terminal end:
an extracellular antigen-binding domain of sequence SEQ ID NO:3,
optionally a hinge domain of sequence SEQ ID NO:4,
a transmembrane domain of sequence SEQ ID NO:5,
a first intracellular signaling domain of sequence SEQ ID NO:6, fused, preferably directly, to a second intracellular signaling domain of sequence SEQ ID NO:7.

The sequences of the application may be summarized as follows:

SEQUENCES

| | | Sequence |
|---|---|---|
| IL-2 | nucleic acid sequence | atggggatccttcccagccctgggatgcctgcgctgctctccctcgt |
| (DNA) | SEQ ID NO: 1 | gagccttctctccgtgctgctgatgggttgcgtagctgaaaccggtg |
| | | cccccaccagctccacaaagaagacccagctgcagctggagca |
| | | cctgctgctggacctgcagatgatcctgaacggcatcaacaattac |
| | | aagaatccaaagctgacacggatgctgaccttcaagttttatatgcc |
| | | caagaaggccacagagctgaagcacctgcagtgcctggaggag |
| | | gagctgaagcctctggaggaggtgctgaacctggcccagtccaa |
| | | gaatttccacctgcggccaagagacctgatctctaacatcaatgtg |
| | | atcgtgctggagctgaagggcagcgagaccaccttcatgtgcgag |
| | | tacgccgatgagaccgccacaatcgtggagttcctgaacaggtgg |
| | | atcaccttttgtcagtccatcatctctaccctgacatga |
| IL-2 | amino acid sequence | MGILPSPGMPALLSLVSLLSVLLMGCVAETGAPT |
| (AA) | SEQ ID NO: 2 | SSTKKTQLQLEHLLLDLQMILNGINNYKNPKLTR |
| | | MLTFKFYMPKKATELKHLQCLEEELKPLEEVLNL |
| | | AQSKNFHLRPRDLISNINVIVLELKGSETTFMCE |
| | | YADETATIVEFLNRWITFCQSIISTLT |
| CAR | extracellular antigen- | MALPVTALLLPLALLLHAARPDIQMTQTTSSLSA |
| (AA) | binding domain of | SLGDRVTISCRASQDISKYLNWYQQKPDGTVKL |
| | sequence | LIYHTSRLHSGVPSRFSGSGSGTDYSLTISNLEQ |
| | SEQ ID NO: 3 | EDIATYFCQQGNTLPYTFGGGTKLEITGGGGSG |
| | | GGGSGGGGSEVKLQESGPGLVAPSQSLSVTCT |
| | | VSGVSLPDYGVSWIRQPPRKGLEWLGVIWGSE |
| | | TTYYNSALKSRLTIIKDNSKSQVFLKMNSLQTDD |
| | | TAIYYCAKHYYYGGSYAMDYWGQGTSVTVSS |
| | hinge domain of sequence | TTTPAPRPPTPAPTIASQPLSLRPEACRPAAGG |
| | SEQ ID NO: 4 | AVHTRGLDFACD |
| | transmembrane domain of | IYIWAPLAGTCGVLLLSLVITLYC |
| | sequence | |
| | SEQ ID NO: 5 | |
| | first intracellular | KKVAKKPTNKAPHPKQEPQEINFPDDLPGSNTA |
| | signaling | APVQETLHGCQPVTQEDGKESRISVQERQ |
| | domain of sequence | |
| | (CD40) | |
| | SEQ ID NO: 6 | |
| | second intracellular | RVKFSRSADAPAYKQGQNQLYNELNLGRREEY |
| | signaling domain of | DVLDKRRGRDPEMGGKPRRKNPQEGLYNELQ |
| | sequence (human | KDKMAEAYSEIGMKGERRRGKGHDGLYQGLST |
| | CD3zeta) | ATKDTYDALHMQALPPR |
| | SEQ ID NO: 7 | |
| | STING intracellular | KGLAPAEISAVCEKGNFNVAHGLAWSYYIGYLR |
| | signaling domain of | LILPELQARIRTYNQHYNNLLRGAVSQRLYILLPL |
| | sequence | DCGVPDNLSMADPNIRFLDKLPQQTGDRAGIKD |
| | SEQ ID NO: 8 | RVYSNSIYELLENGQRAGTCVLEYATPLQTLFA |
| | | MSQYSQAGFSREDRLEQAKLFCRTLEDILADAP |
| | | ESQNNCRLIAYQEPADDSSFSLSQEVLRHLRQE |
| | | EKEEVTVGSLKTSAVPSTSTMSQEPELLISGME |
| | | KPLPLRTDFS |
| CAR2 | Plasmid nucleic acid | caatcaacctctggattacaaaatttgtgaaagattgactgg |
| bands | sequence of SEQ ID NO: 9 | tattcttaactatgttgctccttttacgctatgtggatacgctgctttaatg |
| | comprising IL-2 nucleic | cctttgtatcatgctattgcttcccgtatggctttcattttctcctccttgtat |
| | acid sequence and STING | aaatcctggttgctgtctctttatgaggagttgtggcccgttgtcaggc |
| | nucleic acid sequence | aacgtggcgtggtgtgcactgtgtttgctgacgcaacccccactggt |
| | | tggggcattgccaccacctgtcagctcctttccgggactttcgctttcc |
| | | ccctccctattgccacggcggaactcatcgccgcctgccttgcccg |
| | | ctgctggacaggggctcggctgttgggcactgacaattccgtggtgt |
| | | tgtcggggaaatcatcgtcctttccttggctgctcgcctgtgttgccac |
| | | ctggattctgcgcgggacgtccttctgctacgtcccttcggccctcaa |
| | | tccagcggaccttccttcccgcggcctgctgccggctctgcggcctc |
| | | ttccgcgtcttcgccttcgccctcagacgagtcggatctccctttgggc |
| | | cgcctccccgcctggtacctttaagaccaatgacttacaaggcagc |
| | | tgtagatcttagccactttttaaaagaaaaggggggactggaaggg |
| | | ctaattcactcccaacgaaaataagatctgctttttgcttgtactgggt |
| | | ctctctggttagaccagatctgagcctgggagctctctggctaacta |
| | | gggaacccactgcttaagcctcaataaagcttgccttgagtgcttca |
| | | agtagtgtgtgcccgtctgttgtgtgactctggtaactagagatccctc |
| | | agacccttttagtcagtgtggaaaatctctagcagtagtagttcatgtc |
| | | atcttattattcagtatttataacttgcaaagaaatgaatatcagagag |
| | | tgagaggaacttgtttattgcagcttataatggttacaaataaagcaa |
| | | tagcatcacaaatttcacaaataaagcatttttttcactgcattctagtt |
| | | gtggtttgtccaaactcatcaatgtatcttatcatgtctggctctagctat |
| | | cccgcccctaactccgcccagttccgcccattctccgccccatggct |
| | | gactaattttttttatttatgcagaggccgaggccgcctcggcctctga |
| | | gctattccagaagtagtgaggaggcttttttggaggcctagacttttg |
| | | cagagacggcccaaattcgtaatcatggtcatagctgtttcctgtgtg |
| | | aaattgttatccgctcacaattccacacaacatacgagccggaagc |
| | | ataaagtgtaaagcctggggtgcctaatgagtgagctaactcacat |
| | | taattgcgttgcgctcactgcccgctttccagtcgggaaacctgtcgt |
| | | gccagctgcattaatgaatcggccaacgcgcggggagaggcggt |
| | | ttgcgtattgggcgctcttccgcttcctcgctcactgactcgctgcgctc |
| | | ggtcgttcggctgcggcgagcggtatcagctcactcaaaggcggt |
| | | aatacggttatccacagaatcaggggataacgcaggaaagaac |
| | | atgtgagcaaaaggccagcaaaaggccaggaaccgtaaaaag |
| | | gccgcgttgctggcgtttttccataggctccgcccccctgacgagca |
| | | tcacaaaaatcgacgctcaagtcagaggtggcgaaacccgaca |
| | | ggactataaagataccaggcgtttccccctggaagctccctcgtgc |
| | | gctctcctgttccgaccctgccgcttaccggatacctgtccgcctttct |
| | | cccttcgggaagcgtggcgctttctcatagctcacgctgtaggtatct |
| | | cagttcggtgtaggtcgttcgctccaagctgggctgtgtgcacgaac |
| | | cccccgttcagcccgaccgctgcgccttatccggtaactatcgtcttg |
| | | agtccaacccggtaagacacgacttatcgccactggcagcagcc |
| | | actggtaacaggattagcagagcgaggtatgtaggcggtgctaca |
| | | gagttcttgaagtggtggcctaactacggctacactagaaggacag |
| | | tatttggtatctgcgctctgctgaagccagttaccttcggaaaaagag |
| | | ttggtagctcttgatccggcaaacaaaccaccgctggtagcggtgg |
| | | tttttttgtttgcaagcagcagattacgcgcagaaaaaaaggatctc |
| | | aagaagatcctttgatcttttctacggggtctgacgctcagtggaacg |
| | | aaaactcacgttaagggattttggtcatgagattatcaaaaaggatc |
| | | ttcacctagatccttttaaattaaaaatgaagttttaaatcaatctaaa |
| | | gtatatatgagtaaacttggtctgacagttaccaatgcttaatcagtg |
| | | aggcacctatctcagcgatctgtctatttcgttcatccatagttgcctga |
| | | ctccccgtcgtgtagataactacgatacgggagggcttaccatctg |
| | | gccccagtgctgcaatgataccgcgagacccacgctcaccggctc |
| | | cagatttatcagcaataaaccagccagccggaagggccgagcg |
| | | cagaagtggtcctgcaactttatccgcctccatccagtctattaattgtt |
| | | gccgggaagctagagtaagtagttcgccagttaatagtttgcgcaa |
| | | cgttgttgccattgctacaggcatcgtggtgtcacgctcgtcgtttggt |
| | | atggcttcattcagctccggttcccaacgatcaaggcgagttacatg |
| | | atcccccatgttgtgcaaaaaagcggttagctccttcggtcctccgat |
| | | cgttgtcagaagtaagttggccgcagtgttatcactcatggttatggc |
| | | agcactgcataattctcttactgtcatgccatccgtaagatgcttttctg |
| | | tgactggtgagtactcaaccaagtcattctgagaatagtgtatgcgg |
| | | cgaccgagttgctcttgcccggcgtcaatacgggataataccgcgc |
| | | cacatagcagaactttaaaagtgctcatcattggaaaacgttcttcg |
| | | gggcgaaaactctcaaggatcttaccgctgttgagatccagttcgat |
| | | gtaacccactcgtgcacccaactgatcttcagcatcttttactttcacc |
| | | agcgtttctgggtgagcaaaaacaggaaggcaaaatgccgcaa |
| | | aaaagggaataagggcgacacggaaatgttgaatactcatactct |
| | | tcctttttcaatattattgaagcatttatcagggttattgtctcatgagcg |
| | | gatacatatttgaatgtatttagaaaaataaacaaataggggttccg |
| | | cgcacatttccccgaaaagtgccacctgacgtctaagaaaccatta |
| | | ttatcatgacattaacctataaaaataggcgtatcacgaggccctttc |
| | | gtctcgcgcgtttcggtgatgacggtgaaaacctctgacacatgca |
| | | gctcccggagacggtcacagcttgtctgtaagcggatgccgggag |
| | | cagacaagcccgtcagggcgcgtcagcgggtgttggcgggtgtc |
| | | ggggctggcttaactatgcggcatcagagcagattgtactgagagt |
| | | gcaccatatgcggtgtgaaataccgcacagatgcgtaaggagaa |
| | | aataccgcatcaggcgccattcgccattcaggctgcgcaactgttg |
| | | ggaagggcgatcggtgcgggcctcttcgctattacgccagctggc |
| | | gaaagggggatgtgctgcaaggcgattaagttgggtaacgccag |
| | | ggttttcccagtcacgacgttgtaaaacgacggccagtgccaagct |
| | | gacgcgtgtagtcttatgcaatactcttgtagtcttgcaacatggtaac |
| | | gatgagttagcaacatgccttacaaggagagaaaaagcaccgtg |
| | | catgccgattggtggaagtaaggtggtacgatcgtgccttattagga |
| | | aggcaacagacgggtctgacatggattggacgaaccactgaattg |
| | | ccgcattgcagagatattgtatttaagtgcctagctcgatacaataaa |
| | | cgggtctctctggttagaccagatctgagcctgggagctctctggct |
| | | aactagggaacccactgcttaagcctcaataaagcttgccttgagt |
| | | gcttcaagtagtgtgtgcccgtctgttgtgtgactctggtaactagaga |
| | | tccctcagacccttttagtcagtgtggaaaatctctagcagtggcgcc |
| | | cgaacagggacctgaaagcgaaagggaaaccagagctctctcg |
| | | acgcaggactcggcttgctgaagcgcgcacggcaagaggcgag |
| | | gggcggcgactggtgagtacgccaaaaattttgactagcggaggc |
| | | tagaaggagagagatgggtgcgagagcgtcagtattaagcgggg |
| | | gagaattagatcgcgatgggaaaaaattcggttaaggccagggg |
| | | gaaagaaaaaatataaattaaaacatatagtatgggcaagcagg |
| | | gagctagaacgattcgcagttaatcctggcctgttagaaacatcag |
| | | aaggctgtagacaaatactgggacagctacaaccatcccttcaga |
| | | caggatcagaagaacttagatcattatataatacagtagcaaccct |
| | | ctattgtgtgcatcaaaggatagagataaaagacaccaaggaag |
| | | ctttagacaagatagaggaagagcaaaacaaaagtaagaccac |
| | | cgcacagcaagcggccactgatcttcagacctggaggaggagat |
| | | atgagggacaattggagaagtgaattatataaatataaagtagtaa |
| | | aaattgaaccattaggagtagcacccaccaaggcaaagagaag |
| | | agtggtgcagagagaaaaaagagcagtgggaataggagctttgt |
| | | tccttgggttcttgggagcagcaggaagcactatgggcgcagcctc |
| | | aatgacgctgacggtacaggccagacaattattgtctggtatagtgc |
| | | agcagcagaacaatttgctgagggctattgaggcgcaacagcatc |
| | | tgttgcaactcacagtctggggcatcaagcagctccaggcaagaa |
| | | tcctggctgtggaaagatacctaaaggatcaacagctcctggggat |
| | | ttggggttgctctggaaaactcatttgcaccactgctgtgccttggaat |
| | | gctagttggagtaataaatctctggaacagattggaatcacacgac |
| | | ctggatggagtgggacagagaaattaacaattacacaagcttaat |
| | | acactccttaattgaagaatcgcaaaaccagcaagaaaagaatg |
| | | aacaagaattattggaattagataaatgggcaagtttgtggaattgg |
| | | tttaacataacaaattggctgtggtatataaaattattcataatgatagt |
| | | aggaggcttggtaggtttaagaatagtttttgctgtactttctatagtga |
| | | atagagttaggcagggatattcaccattatcgtttcagacccacctc |
| | | ccaaccccgaggggacccgacaggcccgaaggaatagaaga |
| | | agaaggtggagagagagacagagacagatccattcgattagtga |
| | | acggatctcgacggttaacttttaaaagaaaaggggggattgggg |
| | | ggtacagtgcaggggaaagaatagtagacataatagcaacaga |
| | | catacaaactaaagaattacaaaaacaaattacaaaaattcaaa |
| | | attttatcgatactagttgaattagtttcactttccagtttcagtttccagttt |
| | | cattttccagtttcattttccagtttcattttcctgatatcctgcaggagctt |
| | | gaataaaatgaatattagaagctgttagaataagagaaaatgaca |
| | | gaggaaaactgaaagggagaactgaaagtgggaaattcctctga |
| | | ggcagaaaggaccatcccttataaatagcacaggccatgaagga |
| | | agatcattctcactgcagcctttgacagcctttgcctcatcttgggatc |
| | | caccatggggatccttcccagccctgggatgcctgcgctgctctccc |
| | | tcgtgagccttctctccgtgctgctgatgggttgcgtagctgaaaccg |
| | | gtgcccccaccagctccacaaagaagacccagctgcagctgga |
| | | gcacctgctgctggacctgcagatgatcctgaacggcatcaacaat |
| | | tacaagaatccaaagctgacacggatgctgaccttcaagttttatat |
| | | gcccaagaaggccacagagctgaagcacctgcagtgcctggag |
| | | gaggagctgaagcctctggaggaggtgctgaacctggcccagtc |
| | | caagaatttccacctgcggccaagagacctgatctctaacatcaat |
| | | gtgatcgtgctggagctgaagggcagcgagaccaccttcatgtgc |
| | | gagtacgccgatgagaccgccacaatcgtggagttcctgaacag |
| | | gtggatcaccttttgtcagtccatcatctctaccctgacatgaactagt |
| | | attatgcccagtacatgaccttatgggactttcctacttggcagtacat |
| | | ctacgtattagtcatcgctattaccatggtgatgcggttttggcagtac |
| | | atcaatgggcgtggatagcggtttgactcacggggatttccaagtct |
| | | ccaccccattgacgtcaatgggagtttgttttggcaccaaaatcaac |
| | | gggactttccaaaatgtcgtaacaactccgccccattgacgcaaat |
| | | gggcggtaggcgtgtacggtgggaggtctatataagcagagctcg |
| | | tttagtgaaccgtcagatcgcctggagacgccatccacgctgttttg |
| | | acctccatagaagattctagagctagcgatggccttaccagtgacc |
| | | gccttgctcctgccgctggccttgctgctccacgccgccaggccgg |
| | | acatccagatgacacagactacatcctccctgtctgcctctctggga |
| | | gacagagtcaccatcagttgcagggcaagtcaggacattagtaaa |
| | | tatttaaattggtatcagcagaaaccagatggaactgttaaactcct |
| | | gatctaccatacatcaagattacactcaggagtcccatcaaggttc |
| | | agtggcagtgggtctggaacagattattctctcaccattagcaacct |
| | | ggagcaagaagatattgccacttacttttgccaacagggtaatacg |
| | | cttccgtacacgttcggaggggggaccaagctggagatcacaggt |
| | | ggcggtggctcgggcggtggtgggtcgggtggcggcggatctga |
| | | ggtgaaactgcaggagtcaggacctggcctggtggcgccctcac |
| | | agagcctgtccgtcacatgcactgtctcaggggtctcattacccgac |
| | | tatggtgtaagctggattcgccagcctccacgaaagggtctggagt |
| | | ggctgggagtaatatggggtagtgaaaccacatactataattcagc |
| | | tctcaaatccagactgaccatcatcaaggacaactccaagagcca |
| | | agttttcttaaaaatgaacagtctgcaaactgatgacacagccattta |
| | | ctactgtgccaaacattattactacggtggtagctatgctatggacta |
| | | ctggggccaaggaacctcagtcaccgtctcctcaaccacgacgcc |
| | | agcgccgcgaccaccaacaccggcgcccaccatcgcgtcgcag |
| | | cccctgtccctgcgcccagaggcgtgccggccagcggcggggg |
| | | gcgcagtgcacacgagggggctggacttcgcctgtgatatctacat |
| | | ctgggcgcccttggccgggacttgtggggtccttctcctgtcactggtt |
| | | atcaccctttactgcaagggcctggccccagctgagatctctgcagt |
| | | gtgtgaaaaagggaatttcaacgtggcccatgggctggcatggtc |
| | | atattacatcggatatctgcggctgatcctgccagagctccaggccc |
| | | ggattcgaacttacaatcagcattacaacaacctgctacggggtgc |
| | | agtgagccagcggctgtatattctcctcccattggactgtggggtgc |
| | | ctgataacctgagtatggctgaccccaacattcgcttcctggataaa |
| | | ctgccccagcagaccggtgaccgtgctggcatcaaggatcgggttt |
| | | acagcaacagcatctatgagcttctggagaacgggcagcgggcg |
| | | ggcacctgtgtcctggagtacgccacccccttgcagactttgtttgcc |
| | | atgtcacaatacagtcaagctggctttagccgggaggataggcttg |
| | | agcaggccaaactcttctgccggacacttgaggacatcctggcag |
| | | atgcccctgagtctcagaacaactgccgcctcattgcctaccagga |
| | | acctgcagatgacagcagcttctcgctgtcccaggaggttctccgg |
| | | cacctgcggcaggaggaaaaggaagaggttactgtgggcagctt |
| | | gaagacctcagcggtgcccagtacctccacgatgtcccaagagc |
| | | ctgagctcctcatcagtggaatggaaaagcccctccctctccgcac |
| | | ggatttctcttgagtcga |
| CAR | STING nucleic acid | aagggcctggccccagctgagatctctgcagtgtgtgaaaaaggg |
| (DNA) | sequence of SEQ ID | aatttcaacgtggcccatgggctggcatggtcatattacatcggatat |
| | NO: 10 | ctgcggctgatcctgccagagctccaggcccggattcgaacttaca |
| | | atcagcattacaacaacctgctacggggtgcagtgagccagcgg |
| | | ctgtatattctcctcccattggactgtggggtgcctgataacctgagta |
| | | tggctgaccccaacattcgcttcctggataaactgccccagcagac |
| | | cggtgaccgtgctggcatcaaggatcgggtttacagcaacagcat |
| | | ctatgagcttctggagaacgggcagcgggcgggcacctgtgtcct |
| | | ggagtacgccacccccttgcagactttgtttgccatgtcacaataca |
| | | gtcaagctggctttagccgggaggataggcttgagcaggccaaac |
| | | tcttctgccggacacttgaggacatcctggcagatgcccctgagtct |
| | | cagaacaactgccgcctcattgcctaccaggaacctgcagatgac |
| | | agcagcttctcgctgtcccaggaggttctccggcacctgcggcagg |
| | | aggaaaaggaagaggttactgtgggcagcttgaagacctcagcg |
| | | gtgcccagtacctccacgatgtcccaagagcctgagctcctcatca |
| | | gtggaatggaaaagcccctccctctccgcacggatttctcttga |
| CAR | STINGtz amino acid | KGLAPAEISAVCEKGNFNVAHGLAWSYYIGYLR |
| (AA) | sequence of SEQ ID | LILPELQARIRTYNQHYNNLLRGAVSQRLYILLPL |
| | NO: 11 | DCGVPDNLSMADPNIRFLDKLPQQTGDRAGIKD |
| | | RVYSNSIYELLENGQRAGTCVLEYATPLQTLFA |
| | | MSQYSQAGFSREDRLEQAKLFCRTLEDILADAP |
| | | ESQNNCRLIAYQEPADDSSFSLSQEVLRHLRQE |
| | | EKEEVTVGSLKTSAVPSTSTMSQEPELLISGME |
| | | KPLPLRTDPVETTDYFNPSSAMKQN |
| CAR | STINGtz nucleic acid of | AAGGGCCTGGCCCCAGCTGAGATCTCTGCAG |
| (DNA) | sequence SEQ ID NO: 12 | TGTGTGAAAAAGGGAATTTCAACGTGGCCCAT |
| | | GGGCTGGCATGGTCATATTACATCGGATATCT |
| | | GCGGCTGATCCTGCCAGAGCTCCAGGCCCG |
| | | GATTCGAACTTACAATCAGCATTACAACAACC |
| | | TGCTACGGGGTGCAGTGAGCCAGCGGCTGTA |
| | | TATTCTCCTCCCATTGGACTGTGGGGTGCCTG |
| | | ATAACCTGAGTATGGCTGACCCCAACATTCGC |
| | | TTCCTGGATAAACTGCCCCAGCAGACCGGTG |
| | | ACCGTGCTGGCATCAAGGATCGGGTTTACAG |
| | | CAACAGCATCTATGAGCTTCTGGAGAACGGG |
| | | CAGCGGGCGGGCACCTGTGTCCTGGAGTAC |
| | | GCCACCCCCTTGCAGACTTTGTTTGCCATGTC |
| | | ACAATACAGTCAAGCTGGCTTTAGCCGGGAG |
| | | GATAGGCTTGAGCAGGCCAAACTCTTCTGCC |
| | | GGACACTTGAGGACATCCTGGCAGATGCCCC |
| | | TGAGTCTCAGAACAACTGCCGCCTCATTGCCT |
| | | ACCAGGAACCTGCAGATGACAGCAGCTTCTC |
| | | GCTGTCCCAGGAGGTTCTCCGGCACCTGCGG |
| | | CAGGAGGAAAAGGAAGAGGTTACTGTGGGCA |
| | | GCTTGAAGACCTCAGCGGTGCCCAGTACCTC |
| | | CACGATGTCCCAAGAGCCTGAGCTCCTCATC |
| | | AGTGGAATGGAAAAGCCCCTCCCTCTCCGCA |
| | | CGGATCCTGTGGAGACCACCGATTATTTTAAC |
| | | CCATCTAGCGCAATGAAACAAAACTAA |
| CAR | Promoter promX of | tgaattagtttcactttccagtttcagtttccagtttcattttccagtttcattt |
| (DNA) | sequence SEQ ID NO: 13 | tccagtttcattttcctgatatcctgcaggagcttgaataaaatgaatat |
| | | tagaagctgttagaataagagaaaatgacagaggaaaactgaa |
| | | agggagaactgaaagtgggaaattcctctgaggcagaaaggac |
| | | catcccttataaatagcacaggccatgaaggaagatcattctcact |
| | | gcagcctttgacagcctttgcctcatcttg |
| CAR | Promoter NF-KB of | ggggactttccgggaatttccggggactttccgggaatttccgggaa |
| (DNA) | sequence SEQ ID NO: 14 | tttccggggactttccgggaatttccggggactttccgggaatttcca |
| | | gatctggcctcggcggccaagcttgctagcggggggctataaaag |
| | | ggggtgggggcgttcgt |

### FIGURES

FIG. 1. Lentiviral transduction of monocytes to obtain IL-2 producing macrophages
(A) Untransduced macrophages and macrophages transduced with a control or an IL-2-coding lentiviral vector were harvested at day 10, permeabilized, stained with an anti-IL-2-PE Ab and analyzed by FACS. Left: representative histograms of PE fluorescence. Right: summary of results for multiple donors. MFI ratios are shown compared to untransduced
macrophages.
(B) Quantification of IL-2 in supernatant of approximately 300 000 macrophages/mL cultured for 72 h. Each dot represents one donor. Bars represent the mean+/−SD.
(C) IL-2 concentration present in the SN of macrophages as a function of time post-transduction.

FIG. 2. Transduced macrophages secrete functional IL-2
A. STAT5 phosphorylation detected by flow cytometry in different T cell subsets after 10 min of contact with supernatant (SN), or (B) with recombinant human IL-2 (hlL-2) mean+/−SD. N=3 donors. (C) IL-2-macrophages (IL-2-MØ) were seeded 24 h before adding indicated T cells subset at various ratios for 10 min. Mean of two technical replicates+/−SD. T_{conv}: CD3⁺ CD4⁺ CD25⁻ FoxP3⁻. T_{reg}: CD3⁺ CD4⁺ CD25⁺ FoxP3⁺. CD8 T cells: CD3⁺ CD4⁻.

FIG. 3. IL-2-monocytes induce in vivo tumor regression
(A) NSG mice were injected s.c. with 5 10⁶ MDA-MB-231 CD19⁺GFP⁺ cells. Half of the mice were injected i.v. with CD14− PBMCs (equivalent to 7. 10⁶ cells). Mice were left untreated or injected intra-tumorally (i.t.) with IL-2-Mono or control monocytes. (B) Body weight measured over 35 days. (C) Tumor size measured with a caliper over 35 days.

FIG. 4. Flow cytometry analyses of the spleen and tumors from mice having received cellular therapies

Mice from the experiment depicted in FIG. 3 were sacrificed at day 35 post tumor injection. Their spleen and remaining tumor were harvested, stained for the indicated markers, and analyzed by flow cytometry to determine the fraction of human cells and tumor cells present in these preparations. % of CD3+ cells were not represented for mice with less than 1% of CD45+ cells.

FIG. 5. Tumor growth control capacity of CAR-MØ in 3D

Purified CD14+ cells were transduced with lentivectors encoding the indicated CAR constructs, cultured for 6 days in the presence of M-CSF, and without any antibiotic selection. At day −3, 10³ MDA-MB-231 cells (MDA-GFP-CD19) were seeded in Ultra-Low Attachment 96-well plates, resulting in the formation of tumor spheroids growing in 3D. 3 days later, 2.10³ untransduced macrophages (UTD) or CAR-macrophage (CAR-STOP, CAR-CD3z or CAR-STINGtz) were added to established spheroids. GFP intensity representing spheroid growth of MDA-GFP-CD19 tumor cells was followed by time-lapse microscopy every 3 h for 168 h. The means of GFP intensity calculated from spheroids in triplicates+/−SD are displayed.

FIG. 6. Antigen stimulation of CAR Macrophages induces secretion of proinflammatory cytokines and interferons

Purified CD14+ cells were transduced with lentivectors encoding the indicated CAR constructs cultured for 6 days in the presence of M-CSF and without any antibiotic selection. Macrophages were recovered and seeded alone or with A549 tumor cells expressing CD19+ or not for 24 h. Quantification of cytokines was performed by Legendplex in the supernatant of the indicated co-cultures. Untransduced (UTD) macrophages or macrophages transduced with CAR-STOP, CAR-CD3z or CAR-STINGtz were cultured with media alone (0), A549 or A549-CD19+ cells, at a 1:1 E:T ratio for 24 h. Each dot represents one donor.

FIG. 7. Schematic representation of the CAR2bands proviruses

FIG. 8. Antigen stimulation of CAR2bands Macrophages induces type I interferon response and production of the payload IL2

Purified CD14+ cells were transduced with lentivectors encoding the indicated CAR2bands construct, cultured for 3 days in the presence of M-CSF and without any antibiotic selection. Macrophages were recovered and seeded alone or with A549 tumor cells expressing CD19+ or not for 24 h. Quantification of the indicated cytokines was performed by CBA in the supernatant of the indicated co-cultures. Untransduced (UTD) macrophages or macrophages transduced with IL2-STOP or IL2-STING were cultured with media alone (no tumor), A549 or A549-CD19+ cells. CAR Macrophages were cultured at a 1:1 E:T ratio for 24 h. Each dot represents one donor.

### EXAMPLES

### Example 1: Engineering Autologous Monocytes Expressing IL-2 and/or CAR-STING and its Effect on Anti-Tumor Immunity

### Materials and Methods

### Cell Lines

MDA-MB-231 human breast adenocarcinoma cell line was maintained in RPMI complete medium (Gibco™ Roswell Park Memorial Institute 1640 complemented with 10% fetal calf serum and 1% Gibco™ Penicillin-Streptomycin (Thermofischer)). HEK 293 FT cells were maintained in DMEM complete medium (Gibco™ Dulbecco's Modified Eagle Medium complemented with 10% fetal calf serum and 1% Penicillin-Streptomycin).

MDA-MB-231-GFP and MDA-MB-231-GFP-CD19 were obtained by lentiviral transduction with pWPXLd-GFP coding for GFP and with pCDH1-CD19 coding for hCD19. Transduced cell lines were FACS sorted to obtain homogenous cell populations.

### Primary Cells

Peripheral blood mononuclear cells (PBMC) were separated from plasmapheresis residues using Ficoll-Paque (GE Healthcare). Informed consent was obtained from all donors, and samples were deidentified prior to use in the study. Monocytes were isolated by CD14⁺ positive selection using CD14magnetic microbeads (Miltenyi 130-050-201) and CD3+ T cells by negative selection using (Miltenyi 130-096-535).

### Plasmid Construction and Virus Production

CAR constructs and human IL-2 (hlL-2) were cloned into pCDH1 lentiviral vector containing a puromycin resistance gene under the control of an EF1α promoter.

Lentivirus were produced in HEK293 FT cells. Lentiviral vectors were co transfected with psPAX2 (2nd generation lentiviral packaging plasmid) and pMD2.G (encoding VSV-G) using PEI MAX® (Polysciences). Vpx-VLPs were produced in HEK293FT cells by transfection of pSIV3 and pMD2.G (S. Bobadilla et al., 2013)

After 18 h, the media was replaced with fresh media to remove transfection reagent. Supernatants containing lentivector were collected 24 h after medium change and filtered with a 0.45 μm filter.

### Monocyte Transduction and Differentiation

CD14⁺ cells were transduced with lentivectors in presence of Vpx-VLPs and 4 μg/mL protamine. Monocytes were then allowed to differentiate in macrophages for 10 days in macrophage medium (RPMI+5% fetal calf serum+5% human serum+1% Penicillin-Streptomycin) with 50 ng/mL M-CSF in Corning© 100 mm Not TC-treated Culture Dish.

### Intracellular FACS

Human primary macrophages were treated with 1/1000 GolgiPlug™ (BD Biosciences) for 4 h. Macrophages were detached with Accutase, fixed and permeabilized with BD Cytofix/Cytoperm™, marked with anti-IL-2-PE antibody and analyzed with BD FACSVerse™ flow cytometer.

### FACS-Based Phaqocytosis Assay

1×10⁵ CAR Macrophages were co-cultured with 1×10⁵ A549-GFP or A549-GFP-CD19 cells for 3 h at 37 °C. Cells were harvested with Accutase and stained with anti-CD45-Alexa700 (Biolegend) antibody in presence of FcBlock™ (BD Biosciences) and analyzed with FACS using a Bio-Rad ZE5. The percent of GFP+ events within the CD45+ population was plotted as the percentage of phagocytosis.

### STAT5 Phosphorylation Assay

Human primary T cells were isolated from PBMC with Pan T Cell Isolation Kit human (Miltenyi Biotec). 200 000 T cells were put in contact with cells, supernatants or recombinant human IL-2 for 10 min before being fixed with paraformaldehyde 2%. Cells were permeabilized by methanol, marked with antibodies (anti-CD3-PECy7, anti-CD4-APCCy7, anti-CD25-PE, anti-FoxP3-Alexa488, anti-pSTAT5-Alexa647) and analyzed with BD FACSVerse™ flow cytometer.

### In Vivo Studies

Schemas of the used xenograft model is shown in the panel of the relevant figure. Cells were injected in 100 μl PBS for both IV, IT, and SC injections. Tumor sized is measured twice a week with a caliper. Mice were weighed weekly and were subject to routine veterinary assessment for signs of overt illness. Animals were killed at experimental termination

### Results

### Generation of Macrophages Expressing IL-2

Human primary monocytes purified from PBMCs were transduced with a lentiviral vector encoding IL-2 under the control of the constitutive CMV promoter. After 7 days of culture in the presence of monocyte colony-stimulating factor (M-CSF), the supernatant was replaced by fresh medium. 3 days later the macrophages were harvested. Cell expression of IL-2 was assayed by intracellular flow cytometry with an anti-IL-2-PE antibody after cell permeabilization. While macrophages untransduced or transduced with a control vector did not express IL-2, the great majority (more than 90%) of macrophages transduced with IL-2 (IL-2-Mφ) expressed IL-2 (FIG. 1A). The supernatant of the IL-2-MØ cultured for 3 days contained 300 ng/ml fo IL-2+/−150 ng/ml, as measured by CBA (FIG. 1B). IL-2 concentration was measured in the supernatant of monocytes as a function of time post-transduction. IL-2-transduced monocytes secreted detectable levels IL-2 as soon as 3 days post transduction (FIG. 1C). The inventors concluded that monocytes transduced with our IL-2 lentivector give rise to macrophages rapidly producing important amounts of IL-2 at high rate (estimation 1 fg IL-2/Mφ).

### IL-2 Produced by IL-2-MØ is Functionally Active on Primary T Lymphocytes

The inventors assayed the functionality of the IL-2 produced by IL-2-MØ by measuring the IL-2-induced phosphorylation of STAT5 that initiates the signaling cascade leading to T cell survival and proliferation. The supernatant of IL-2-MØ was incubated with human primary T cells for 10 min before fixation, permeabilization of the cells, and staining with an antibody specific for phosphorylated STAT5. Total CD3+ T cells freshly purified from PBMC isolated from healthy donors were used in this assay. STAT5 phosphorylation was measured in three T cell subsets: CD4⁺ T_{conv} (conventional T cells, defined as CD3+ CD4+, CD25− Foxp3−), T_{reg} (regulatory T cells, defined as CD3+ CD4+, CD25+, Foxp3+) and CD8⁺ T cells (defined as CD3+CD4−), because they express the IL-2R at different levels, the high affinity IL-2R being mainly expressed by Treg at the steady state. Only the supernatant of IL-2-Mφ triggered the phosphorylation of STAT5 in all populations of T cells to various extents: Treg>T conv>CD8 T cells in agreement with their respective expression of IL-2R chains (FIG. 2A).

In parallel, the inventors titrated in the same assay purified recombinant human IL-2 (hlL-2) and different dilutions of the IL-2-Mφ supernatant (FIG. 2B). The curves obtained with the 3 populations of T cells were similar in both situations with higher rates of STAT5 phosphorylation in T_{reg}, due to their expression of the high affinity IL-2R, as compared to T_{conv} and CD8⁺ T cells. Thus, the IL-2 secreted by IL-2-Mφ was functionally active on all populations of T cells.

The inventors also assessed the potential of IL-2-Mφ more directly, by plating them for 24 h before directly adding primary T cells for 10 min (FIG. 2C). Different ratios of macrophages to T cells were tested, giving rise to nice dose effect curves. The presence of macrophages did not impair the capacity of secreted IL-2 to phosphorylate STAT5 in a dose-dependent manner similarly to hlL-2. Together, our data show that IL-2-Mφ secrete substantial amounts of IL-2 that are capable to activate all populations of primary T cells.

### Anti-Tumor Activity of and IL-2-MØ In Vivo

To evaluate the anti-tumor activity of IL-2-MØ in vivo, the inventors used the human breast carcinoma MDA-MB-231 cell line to generate cells expressing CD19 and GFP by lentiviral transduction. MDA-MB-231 GFP⁺CD19⁺ cells were injected by the subcutaneous route into immunodeficient NSG mice. Half of the mice received at day 10 an intravenous (iv) injection of monocytes-depleted PBMCs (CD14− cells). The proportion of CD3⁺ T cells present in the CD14− fraction was estimated by flow cytometry and the number of CD14-cells injected was adjusted to contain 7.10⁶ CD3⁺ T cells. At day 11, mice received an intratumoral (i.t.) injection of either 10.10⁶ of IL-2-transduced monocytes (Mono-IL-2) or the same number or monocytes transduced with an empty vector (control monocytes). Of note the monocytes were transduced in vitro at day 10, left overnight in the incubator and injected it at day 11, i.e. less than 24 h after being purified and transduced.

The mouse body weight and the growth of the s.c. tumors were regularly monitored over a 35-day period (FIG. 3A). The treatments did not impact the body weight of the mice as compared to untreated control mice, suggesting a lack of a broad toxicity effect (FIG. 3B). The inventors observed that the Mono-IL-2 cells were unable to control tumor growth by themselves and CD14− cells mediated a limited control of tumor growth. In contrast, 5 out of 6 mice which received both CD14− cells and Mono-IL-2 demonstrated a marked reduction in their tumor burden. These data suggest that the injected Mono-IL-2 need to cooperate with CD14− cells (probably T cells) to control tumor growth (FIG. 3C). n=2 experiments.

The inventors concluded that transduction of monocytes with IL-2 encoding vector results in cells able to induce tumor regression in immunodeficient mice partially reconstituted with T cells. Thus, injection of Mono-IL-2 and CD14− cells induces in NSG Mice carrying a human tumor an efficient graft versus tumor reaction.

### Analysis by Flow Cytometry of the Cell Populations Present in the Treated Mice

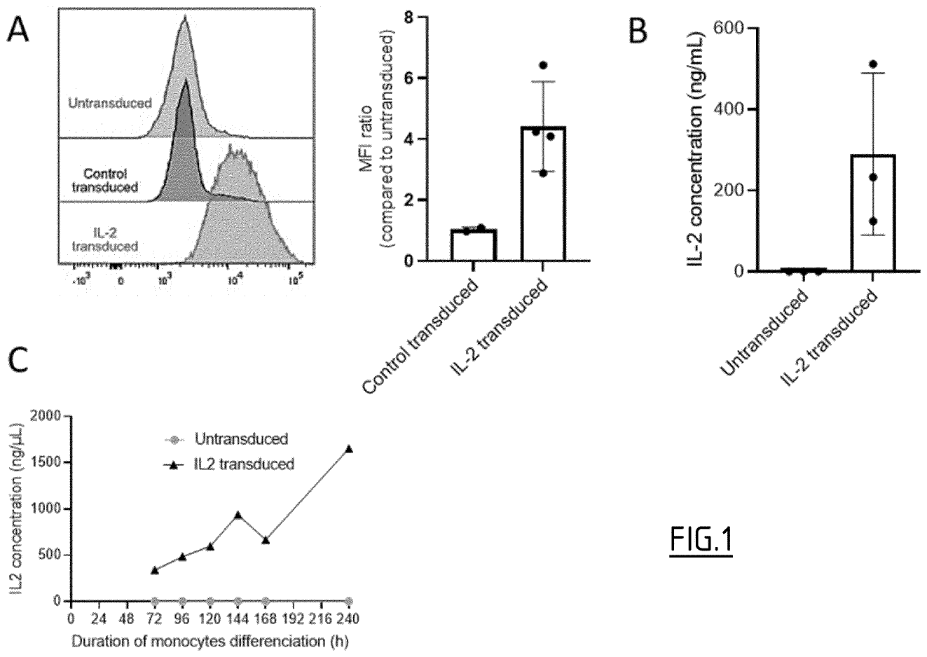

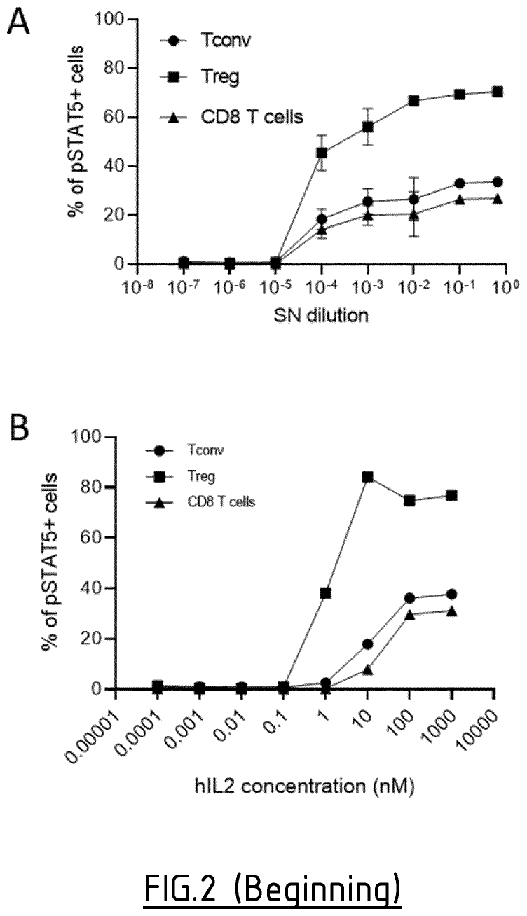

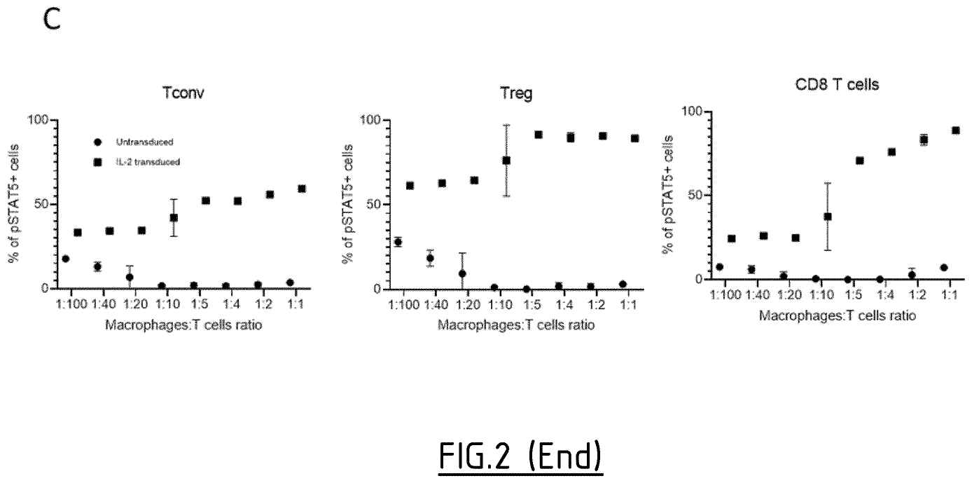

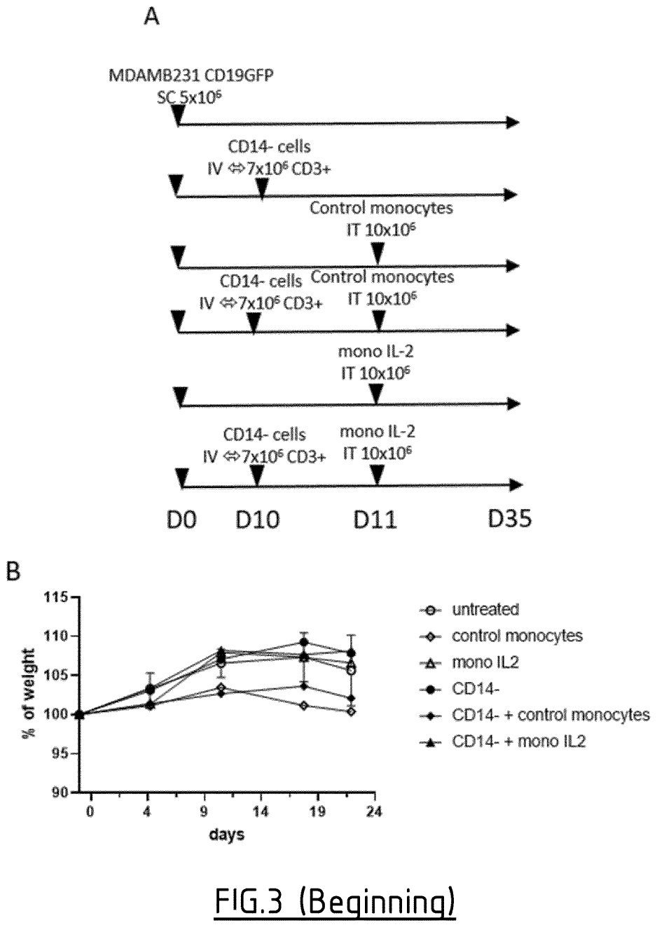

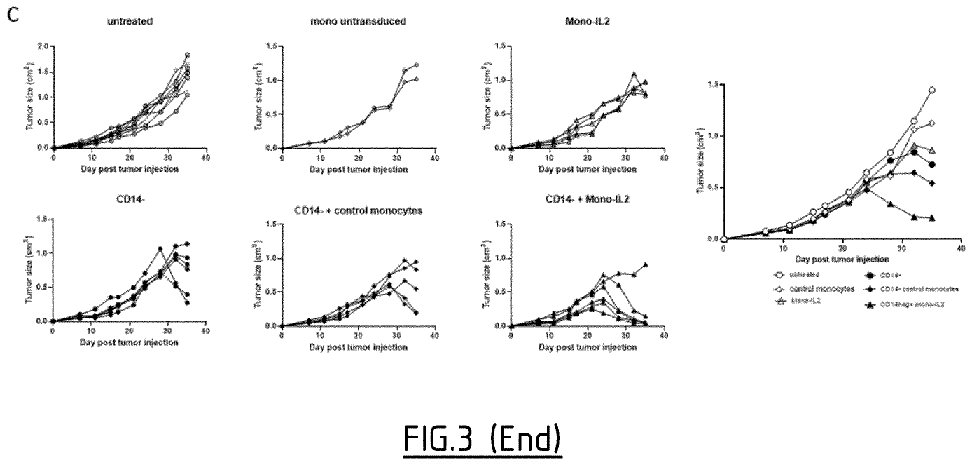

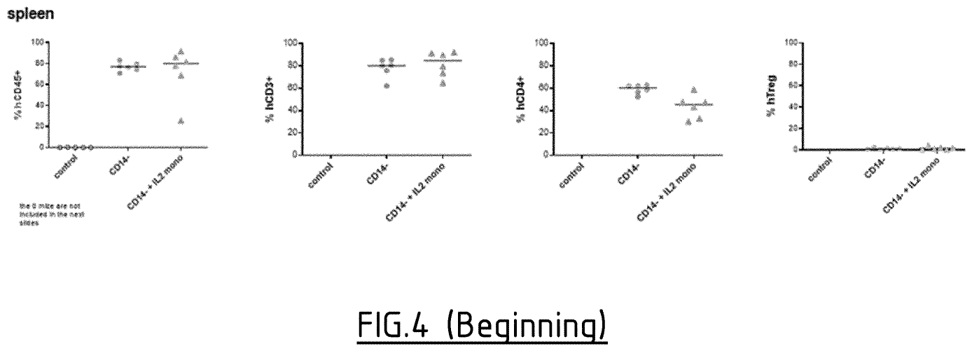

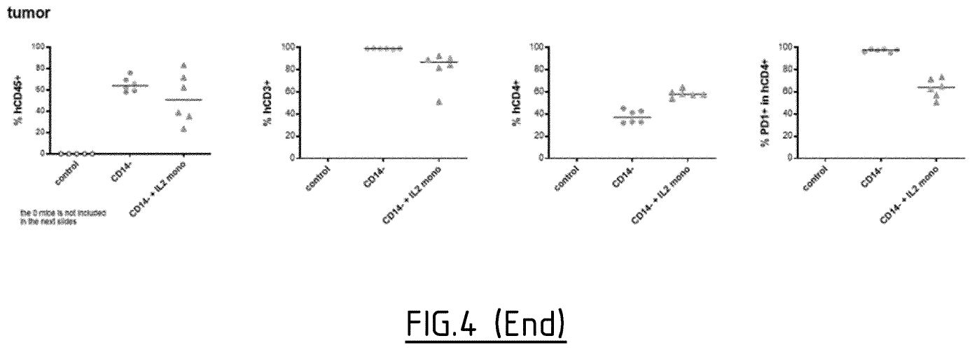

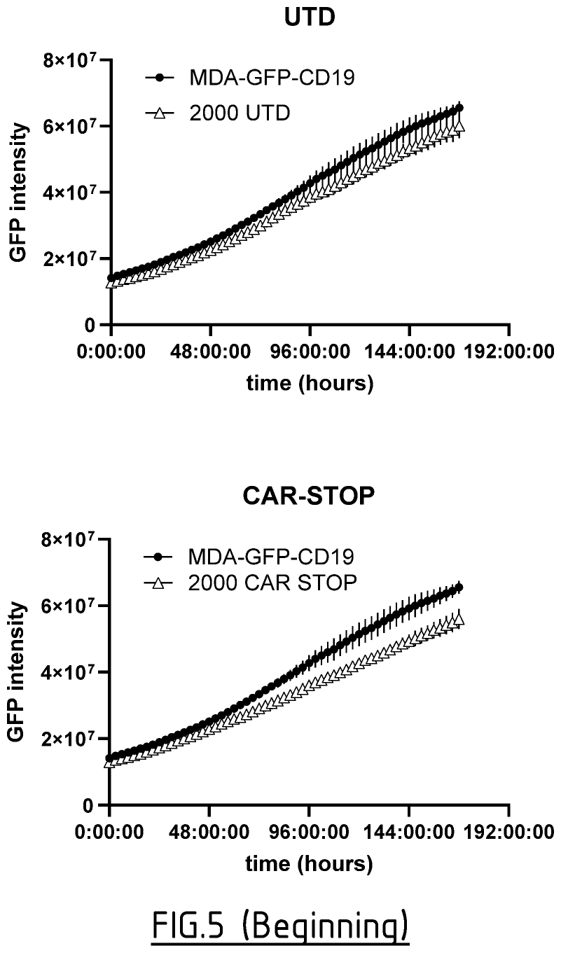

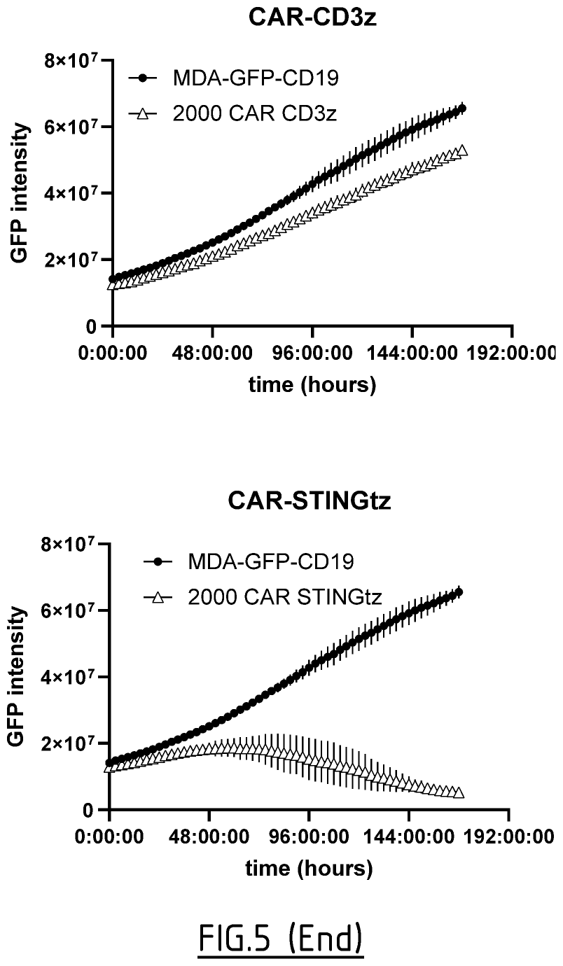

Mice from the experiments described in FIG. 3 were sacrificed at day 35 post tumor injection, their spleen and remaining tumor were harvested, stained, and analyzed by flow cytometry (FIG. 4). Human myeloid cells remained undetectable using a CD64 specific Ab at both locations. Nevertheless, mice injected with Mono-IL-2 contained high levels of human CD45+ cells which were in majority CD3⁺T cells. Of note, the purity of our monocyte preparations were regularly higher than 98% (not shown), indicating that in the group of mice which only received Mono-IL-2 the very few contaminating T cells were probably efficiently expended with the help of the IL-2 produced by the Mono-IL-2 (see panel A in the tumor the % of CD3+ T cells).

Spheroid growth assay of MDA-MB-231 GFP⁺ CD19⁺ cells co-cultured with various CAR-MØ

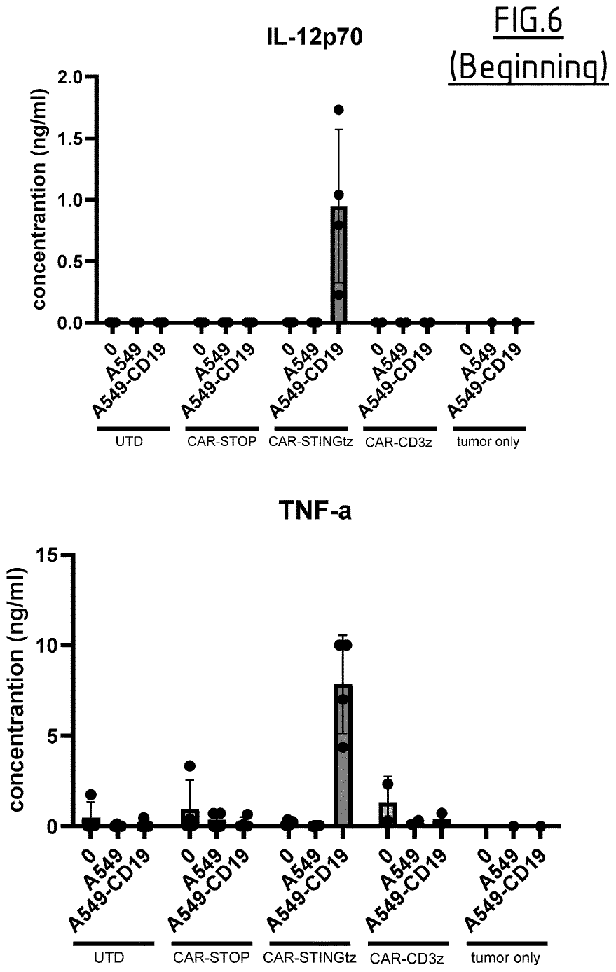

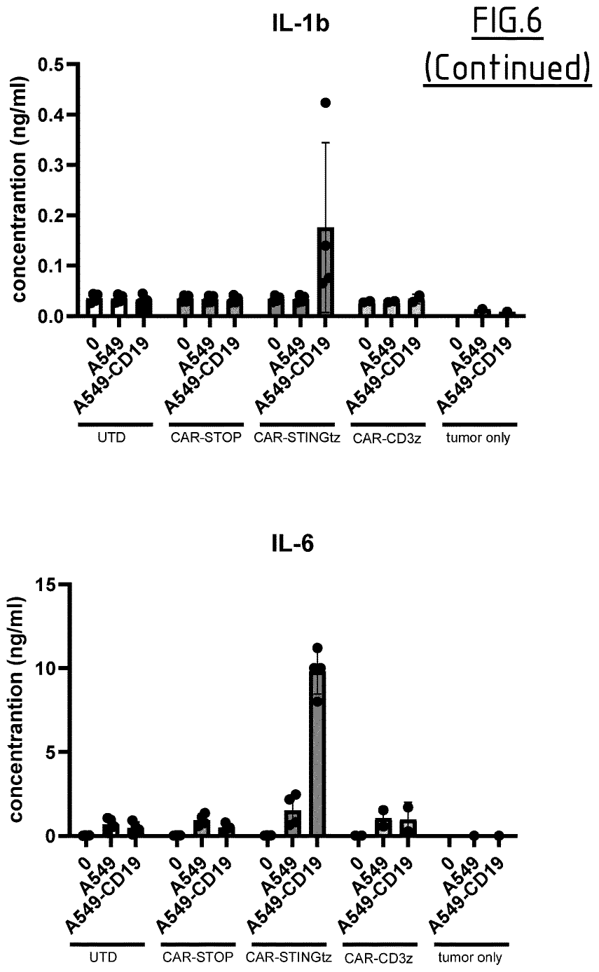

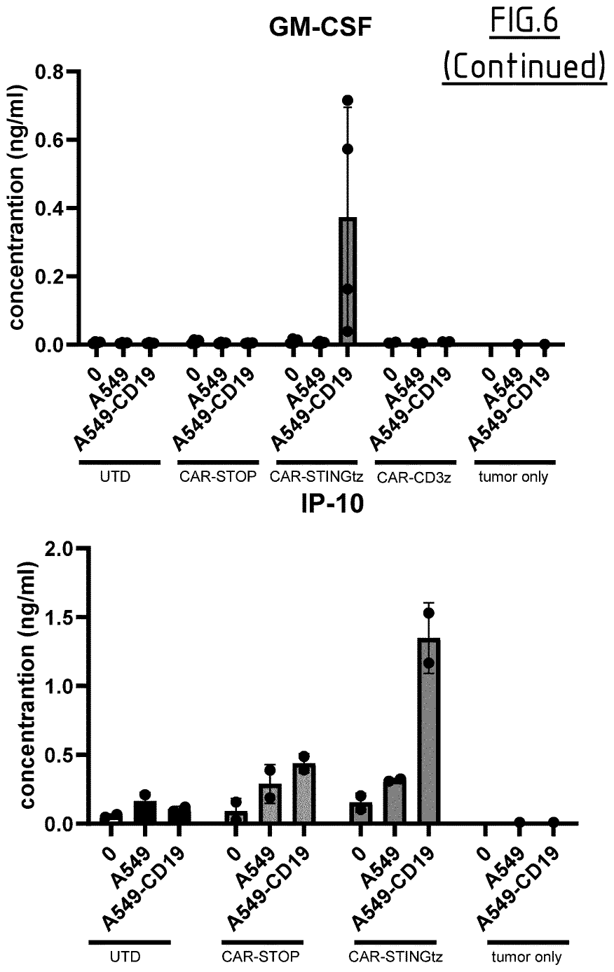

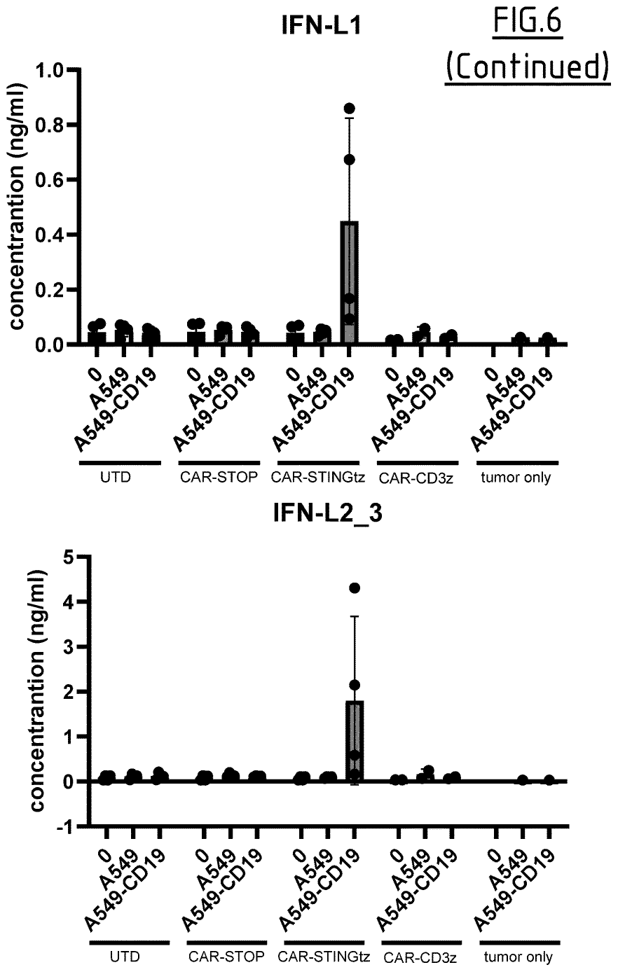

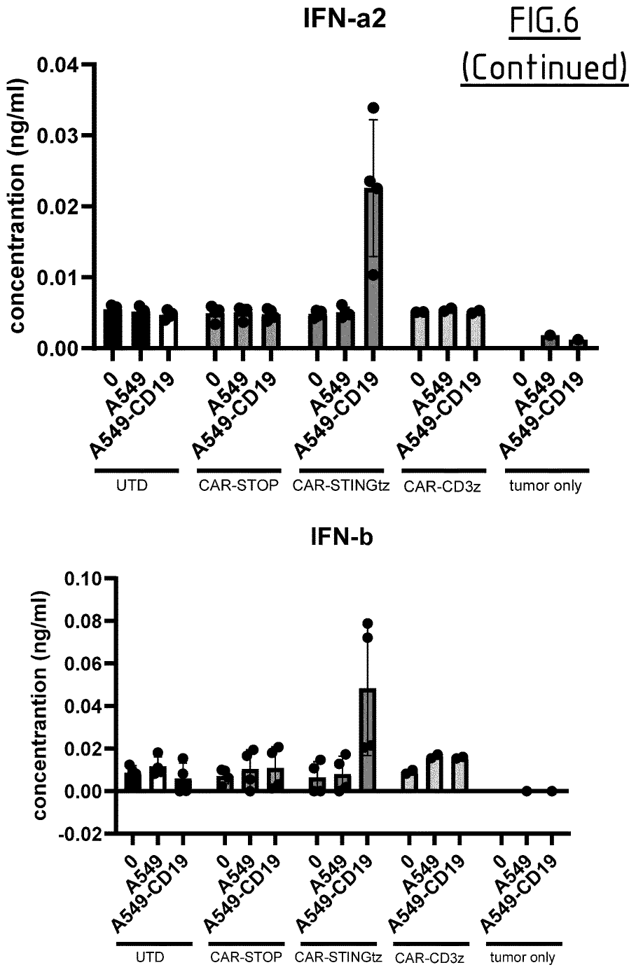

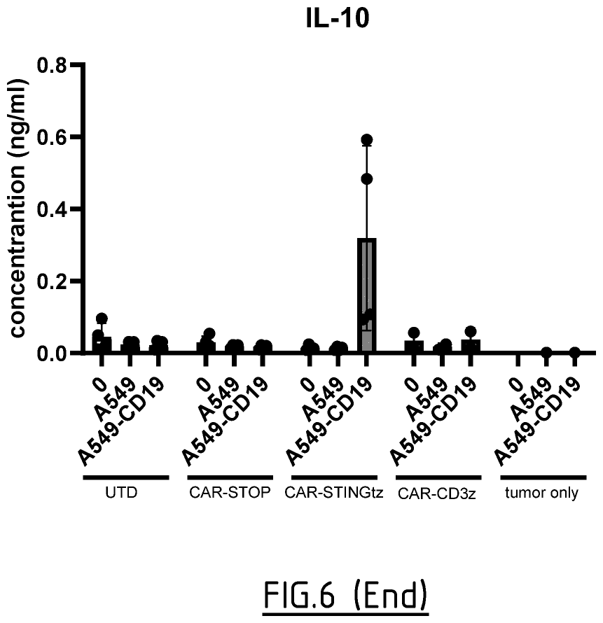

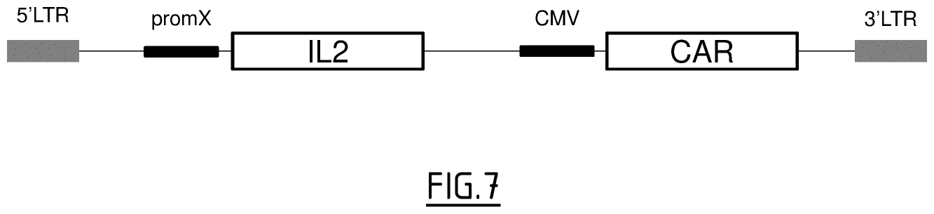

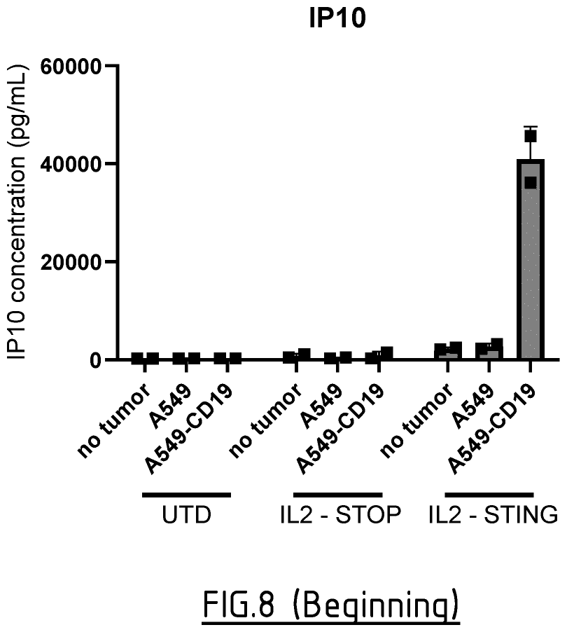

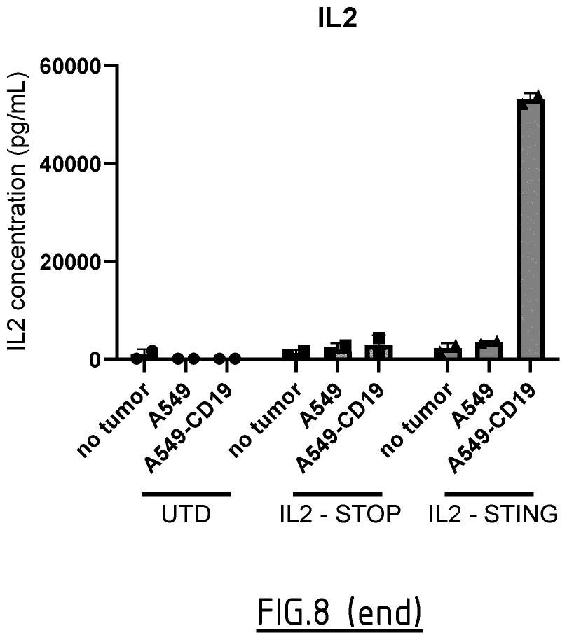

The inventors assayed the capacity of CAR-macrophage (CAR-MØ) to control tumor growth using tumor spheroids of MDA-MB-231 cells expressing GFP and CD19 (MDA-GFP-CD19). 3 days later, untransduced macrophages (UTD) or CAR-MØ (CAR-STOP, CAR-CD3z or CAR-STINGtz) were added to establish spheroids. Spheroids growth is measured by live cell imaging using GFP fluorescence (FIG. 5). The inventors concluded that adding 2000 CAR-STINGtz macrophages to established tumor spheroids (3 days old) induces a very efficient tumor regression that is not observed when using untransduced, CAR-STOP or CAR-CD3z-transduced macrophages.

### Antigen Stimulation of CAR Macrophages Induces Secretion of Proinflammatory Cytokines and Interferons

To evaluate the capacity of CAR-macrophage (CAR-MØ) to produce cytokines in an antigen-dependent manner, CAR-MØ were seeded alone or with A549 tumor cells expressing CD19+ or not for 24 h. Quantification of cytokines was performed in the indicated co-cultures (FIG. 6). The inventors concluded that macrophages expressing the CAR-STINGtz can produce substantial amounts of a panel of cytokines of interest in a strict antigen-dependent manner compared to CAR-STOP or CAR-CD3z. Of note, IL12 (IL-12p70) is essential for T cell priming, GM-CSF promotes myeloid cell survival and activates macrophages, IFNalpha (IFN-a2) and beta (IFN-b) are essential to elicit a robust anti-tumor response locally. IL-1 (IL-1 b) and IL-6 activate T cells but are antagonistic to Treg cells. IL-10 production reflects macrophage activation. TNFalpha (TNF-a) enhances phagocytosis. Thus, the CAR-STINGtz macrophages according to the invention allows to activate, upon CD19+ antigen binding, both the IRF3 and NF-KB pathways, stimulating the production of several cytokines. Importantly, in the absence of the tumor antigen, almost no cytokines are released by the CAR macrophages.

### Antigen Stimulation of CAR2Bands Macrophages Induces Type I Interferon Response And Production of the Payload IL2

The inventors assessed the capacity of CAR2bands macrophages to induce type I interferon response (type I IFN) and to produce IL-2 in an antigen-dependent manner. The structure of the CAR2bands vector is in FIG. 7. Untransduced (UTD) macrophages or CAR2bands macrophages transduced with IL2-STOP or IL2-STING were cultured with media alone (no tumor), A549 or A549-CD19+ cells and cytokines in the supernatant were quantified (FIG. 8). The inventors concluded that macrophages expressing the CAR2bands IL2-STING can produce substantial amounts of IP10 when put in contact with CD19+ expressing cells, revealing that activation of their CAR containing a truncation of STING leads to type I IFN production, which in turn induces the expression of interferon-stimulated genes such as IP10. Moreover, activation of STING also leads to the activation of the minimal promoter driving the expression of the payload IL2. Substantial amounts of IL2 are thus produced in response to antigen exposure.

1. A modified cell, wherein the cell is chosen from myeloid cells, induced pluripotent stem cells (iPS) and hematopoietic stem cells (HSC), and wherein the cell comprises:
(i) either a first vector comprising a sequence coding for at least one cytokine, preferably at least one interleukin, under the control of an inducible or constitutive promoter, and a second vector comprising a sequence coding for a chimeric antigen receptor (CAR); or
(ii) only comprises the first vector, which encodes for both the at least one cytokine, preferably at least one interleukin, and for the CAR.

2. The modified cell according to claim 1, wherein it is a modified myeloid cell, preferably a monocyte, a macrophage or a dendritic cell, and more preferably a monocyte.

3. The modified cell according to claim 1, wherein the cytokine is IL-2, and the sequence coding for IL-2 is the sequence SEQ ID NO:2.

4. The modified cell according to claim 1, wherein the promoter is inducible.

5. The modified cell according to claim 1, wherein said CAR comprises:
an extracellular antigen-binding domain which binds to a tumor antigen or an antigen present on cells of the tumor microenvironment (TME);
optionally a hinge domain;
a transmembrane domain, and
an intracellular signaling domain.

6. The modified cell according to claim 5, wherein the extracellular antigen-binding domain is chosen from CD19, MUC16, MUC1, CA1X, carcinoembryonic antigen (CEA), CD8, CD7, CD 10, CD20, CD22, CD30, CLL1, CD33, CD34, CD38, CD41, CD44, CD49f, CD56, CD74, CD133, CD138, EGP-2, EGP-40, EpCAM, erb-B2,3,4, FBP, Fetal acetylcholine receptor, folate receptor-a, GD2, GD2Ac, GD3, ITER-2, hTERT, IL-13R-a2, K-light chain, KDR, LeY, L1 cell adhesion molecule, MAGE-A1, Mesothelin, ERBB2, MAGEA3, p53, MARTI, GP100, Proteinase 3 (PR1), Tyrosinase, Survivin, EphA2, NKG2D ligands, NY-ESO-1, oncofetal antigen (h5T4), PSCA, PSMA, ROR1, TAG-72, VEGF-R2, WT-1, BCMA, CD123, CD44V6, NKCS1, EGF1R, EGFR-VIII, CD99, CD70, ADGRE2, CCR1, LILRB2, PRAME, CCR4, CD5, CD3, TRBC1, TRBC2, TIM-3, Integrin B7, ICAM-1, CD70, Tim3, CLEC12A, ER, human telomerase reverse transcriptase (hTERT), mouse double minute 2 homolog (MDM2), cytochrome P450 1B1 (CYP1B), HER2/neu, p95HER2, Wilms' tumor gene 1 (WT1), livin, alphafetoprotein (AFP), prostate-specific membrane antigen (PSMA), cyclin (D1), mesothelin, B-cell maturation antigen (BCMA) and tumor-associated calcium signal transducer 2 (TROP2), preferably the extracellular antigen-binding domain is an anti-CD19 binding domain, preferably an anti-CD19 scFV.

7. The modified cell according to claim 5, wherein the intracellular signaling domain is either a first intracellular signaling domain comprising the CD40 cytoplasmic tail, preferably of sequence SEQ ID NO:6, or a first intracellular signaling domain comprising the CD40 cytoplasmic tail, preferably of sequence SEQ ID NO:6 which is fused to a second intracellular signaling domain comprising the CD3zeta intracellular domain, preferably of sequence SEQ ID NO:7.

8. The modified cell according to claim 1, wherein the CAR comprises, from its N-terminal end to its C-terminal end:
an extracellular antigen-binding domain of sequence SEQ ID NO:3,
optionally a hinge domain of sequence SEQ ID NO:4,
a transmembrane domain of sequence SEQ ID NO:5, and
either a first intracellular signaling domain of sequence SEQ ID NO:6, or a first intracellular signaling domain of sequence SEQ ID NO:6 which is fused, preferably directly, to a second intracellular signaling domain of sequence SEQ ID NO:7.

9. The modified cell according to claim 1, wherein said CAR comprises:
an extracellular antigen-binding domain which binds to a tumor antigen or an antigen present on cells of the TME;
optionally a hinge domain,
a transmembrane domain; and
an intracellular signaling domain comprising STING or one of its fragments;
and wherein said modified cell is a myeloid cell.

10. The modified cell according to claim 1, wherein the promoter of the first vector is a cytokine specific promoter, preferably the promoter of interleukin-6 or the promoter of interleukin-8.

11. The modified cell according to claim 1, wherein the promoter of the first vector is an artificial promoter containing response elements that are activated via the activation of the CAR.

12. A modified cell expressing at least one cytokine, preferably an interleukin chosen from IL-10, IL-15, IL-13, IL-7A, IFNalpha, IFNbeta, IFNlambda, IFNgamma, IL-1A, IL-1B, IL-12 and IL-21, wherein the cell is chosen from myeloid cells, induced pluripotent stem cells (iPS) and hematopoietic stem cells (HSC), and wherein the cell comprises a first vector comprising a sequence coding for said cytokine or interleukin under the control of an inducible or constitutive promoter, and optionally a second vector comprising a sequence coding for a chimeric antigen receptor (CAR),
wherein said CAR comprises:
an extracellular antigen-binding domain which binds to a tumor antigen or an antigen present on cells of the tumor microenvironment (TME);
optionally a hinge domain;
a transmembrane domain, and
an intracellular signaling domain.

13. A method for manufacturing a modified myeloid cell, a modified iPS or a modified HSC according to claim 1, the method comprising
providing at least one cell chosen from isolated myeloid cells, iPS and HSC;
transducing said cell with a first vector, preferably a lentiviral vector, comprising a sequence coding for IL-2 or a sequence coding for at least one cytokine, preferably an interleukin chosen from IL-10, IL-15, IL-13, IL-7A, IFNalpha, IFNbeta, IFNlambda, IFNgamma, IL-1A, IL-1B, IL-12 and IL-21, under the control of an inducible or constitutive promoter, preferably under the control of the promoter of interleukin-6 or the promoter of interleukin-8; and
optionally, transducing said cell with a second vector, preferably a lentiviral vector, comprising a nucleic sequence coding for a CAR.

14. A pharmaceutical composition comprising the modified cell according to claim 1 and a pharmaceutical acceptable carrier.

15. A method of treatment of cancer, an autoimmune disease or an inflammatory disease comprising administering to a subject in need thereof the modified cell according to a claim 1.

16. The method of claim 15, wherein the modified cell is a myeloid cell and is autologous of said donor.

17. A method of treatment of cancer, an autoimmune disease or an inflammatory disease comprising the simultaneous, separate or sequential administration to a subject in need thereof of a combined preparation of products containing a modified cell according to claim 1 and a CAR-T cell, or an Immune Checkpoint Inhibitor.

18. A method of treatment of cancer, an autoimmune disease or an inflammatory disease comprising administering to a subject in need thereof the pharmaceutical composition of claim 14, wherein the cancer is preferably a solid tumor.
